SEQUENCE LISTING (51 sequences)

SEQ ID NO: 1 - DNA, 41 bp, Artificial Sequence
  SYNTHESIZED
tccatatggcggccgcctgtcgggaatcgcgttcaggtgga

SEQ ID NO: 2 - DNA, 33 bp, Artificial Sequence
  SYNTHESIZED
atgcatgctgtgagaaaataaagtgctttgtgc

SEQ ID NO: 3 - DNA, 44 bp, Artificial Sequence
  SYNTHESIZED
gcgtcgacgttggcgcgcctgtaacactcgctctggagagttag

SEQ ID NO: 4 - DNA, 41 bp, Artificial Sequence
  SYNTHESIZED
ccacatgtgcggccgcactgaagggctttgtgagagaggta

SEQ ID NO: 5 - DNA, 32 bp, Artificial Sequence
  SYNTHESIZED
cgggatccatgtctaagaagcatattgtgatt

SEQ ID NO: 6 - DNA, 33 bp, Artificial Sequence
  SYNTHESIZED
ttcctaggcttagatgacgttggagttgtgcac

SEQ ID NO: 7 - DNA, 33 bp, Artificial Sequence
  SYNTHESIZED
cgggatccatgctgctgacttacatggaggttc

SEQ ID NO: 8 - DNA, 33 bp, Artificial Sequence
  SYNTHESIZED
ttcctaggttagatggtgttcaggtttcgcatc

SEQ ID NO: 9 - DNA, 27 bp, Artificial Sequence
  SYNTHESIZED
cgggatccatgtccaaggcgaaattcg

SEQ ID NO: 10 - DNA, 34 bp, Artificial Sequence
  SYNTHESIZED
ttcctaggtcactgcgcatcctcaaagtactttc

SEQ ID NO: 11 - DNA, 33 bp, Artificial Sequence
  SYNTHESIZED
gcgtcgacagagaccgggttggcggcgtatttg

SEQ ID NO: 12 - DNA, 39 bp, Artificial Sequence
  SYNTHESIZED
ttggcgcgccgccacctacaagccagattttctatttac

SEQ ID NO: 13 - DNA, 35 bp, Artificial Sequence
  SYNTHESIZED
ttggcgcgccagagaccgggttggcggcgtatttg

SEQ ID NO: 14 - DNA, 33 bp, Artificial Sequence
  SYNTHESIZED
cgggatccatgggcaccatcgaccgagctgagg

SEQ ID NO: 15 - DNA, 33 bp, Artificial Sequence
  SYNTHESIZED
ttcctaggttatcgcatctccttagcagcaggg

SEQ ID NO: 16 - DNA, 33 bp, Artificial Sequence
  SYNTHESIZED
cgggatccatgatgctgaaggctggtaaccgac

SEQ ID NO: 17 - DNA, 34 bp, Artificial Sequence
  SYNTHESIZED
ttcctaggctacttagtgttagaggtcgaaaaag

SEQ ID NO: 18 - DNA, 33 bp, Artificial Sequence
  SYNTHESIZED
cgggatccatggagtctaagctgctgcgaaaca

SEQ ID NO: 19 - DNA, 33 bp, Artificial Sequence
  SYNTHESIZED
ttcctaggctaggacttcacctgagctcgggcc

SEQ ID NO: 20 - DNA, 33 bp, Artificial Sequence
  SYNTHESIZED
cgggatccatgcagcagcgactgaccaagcgac

SEQ ID NO: 21 - DNA, 33 bp, Artificial Sequence
  SYNTHESIZED
ttcctaggttacttagaggaagcagagggcttg

SEQ ID NO: 22 - DNA, 33 bp, Artificial Sequence
  SYNTHESIZED
cgggatccatgtccgacatggagaaggagtctg

SEQ ID NO: 23 - DNA, 33 bp, Artificial Sequence
  SYNTHESIZED
ttcctaggttagatggaggccagctcagcggcg

SEQ ID NO: 24 - DNA, 33 bp, Artificial Sequence
  SYNTHESIZED
cgggatccatgggtgaccgaggctcctctcact

SEQ ID NO: 25 - DNA, 33 bp, Artificial Sequence
  SYNTHESIZED
ttcctaggtcaagatccggatcggattcgtcgg

SEQ ID NO: 26 - DNA, 1740 bp, Artificial Sequence
  SYNTHESIZED
atgtctaagaagcatattgtgattattggagccggagttggaggcaccgctaccgccgcccgactggcccgagagggattcaaggttaccgtcgtggagaagaacgactttggcggtggacgatgctctctgatccaccatcagggacaccgattcgaccagggcccctccctgtacctcatgcctaagtactttgaggatgccttcgctgacctggatgagcgaatccaggaccacctcgagctgctccgatgtgataacaactacaaggttcatttcgacgatggcgagtctattcagctgtcctctgacctcacccgaatgaaggccgagctggatcgagtcgagggtcccctgggattcggccgatttctcgacttcatgaaggagacccacatccattacgagtctggaactctgattgctctcaagaagaacttcgagtcgatttgggacctgatccgaattaagtacgcccccgagattttccgactgcacctcttcggcaaaatctacgatcgagcctccaagtacttcaagaccaagaagatgcgaatggctttcacctttcagactatgtacatgggcatgtccccctacgacgcccctgctgtgtactctctgctccagtacaccgagtttgccgagggtatctggtatccccgaggcggtttcaacatggttgtccagaagctggaggccattgccaagcagaagtacgacgctgagttcatctacaacgcccctgtggctaagattaacaccgacgatgccactaagcaggtgaccggtgttactctggagaacggacacatcattgacgccgatgctgtggtttgcaacgccgacctcgtctacgcttaccataacctgctccccccttgtcgatggacccagaacactctggcctcgaagaagctcacttcgtcctctatctccttctactggtcgatgtccaccaaggttccccagctggacgtccacaacatctttctcgccgaggcttaccaggagtctttcgacgagatttttaaggatttcggactgccctctgaggcttcgttctacgttaacgtcccttctcgaattgacccctcggccgctcctgacggcaaggattccgtgatcgttctcgtccccattggccatatgaagtcgaagaccggtgacgcctccactgagaactaccctgccatggtggataaggctcgaaagatggtcctggccgtgatcgagcgacgactcggaatgtctaactttgctgacctgattgagcacgagcaggtgaacgatcccgccgtttggcagtccaagttcaacctctggcgaggctccatcctgggtctctctcatgacgttctgcaggtcctctggttccgaccttccaccaaggactctactggacgatacgataacctgttctttgtcggcgcttctacccaccccggtactggagtgcctatcgttctggccggttcgaagctcacctccgaccaggtcgtgaagtccttcggaaagactcccaagcctcgaaagattgagatggagaacacccaggctcctctggaggagcctgacgctgagtctacctttcccgtctggttctggctccgagccgctttttgggtcatgttcatgttcttttacttctttccccagtctaacggacagacccctgcctcgtttatcaacaacctgctccccgaggtcttccgagtgcacaactccaacgtcatctaa

SEQ ID NO: 27 - protein, 579 aa, Artificial Sequence
  SYNTHESIZED
MSKKHIVIIGAGVGGTATAARLAREGFKVTVVEKNDFGGGRCSLIHHQGHRFDQGPSLYLMPKYFEDAFADLDERIQDHLELLRCDNNYKVHFDDGESIQLSSDLTRMKAELDRVEGPLGFGRFLDFMKETHIHYESGTLIALKKNFESIWDLIRIKYAPEIFRLHLFGKIYDRASKYFKTKKMRMAFTFQTMYMGMSPYDAPAVYSLLQYTEFAEGIWYPRGGFNMVVQKLEAIAKQKYDAEFIYNAPVAKINTDDATKQVTGVTLENGHIIDADAVVCNADLVYAYHNLLPPCRWTQNTLASKKLTSSSISFYWSMSTKVPQLDVHNIFLAEAYQESFDEIFKDFGLPSEASFYVNVPSRIDPSAAPDGKDSVIVLVPIGHMKSKTGDASTENYPAMVDKARKMVLAVIERRLGMSNFADLIEHEQVNDPAVWQSKFNLWRGSILGLSHDVLQVLWFRPSTKDSTGRYDNLFFVGASTHPGTGVPIVLAGSKLTSDQVVKSFGKTPKPRKIEMENTQAPLEEPDAESTFPVWFWLRAAFWVMFMFFYFFPQSNGQTPASFINNLLPEVFRVHNSNVI

SEQ ID NO: 28 - DNA, 1845 bp, Artificial Sequence
  SYNTHESIZED
atgctgctgacttacatggaggttcatctctactacactctgcctgttctgggcgttctgtcttggctgtcccgaccctactacactgccactgacgctctgaagttcaagtttctgaccctcgttgccttcaccactgcctcggcttgggataactacatcgtctaccacaaggcctggtcttactgccccacctgtgttactgctgtcattggctacgtccctctggaggagtacatgttctttatcattatgaccctgctcactgttgccttcaccaacctcgtcatgcgatggcacctgcattccttctttatccgacccgagacccctgttatgcagtctgtcctggtgcgactcgtccccatcaccgccctgctcattactgcctacaaggcttggcacctcgctgtgcccggaaagcctctgttctacggctcctgcatcctctggtacgcctgtcctgttctggctctgctctggtttggtgccggagagtacatgatgcgacgacccctggccgttctcgtctctattgctctgcctactctgttcctctgctgggtggacgtcgtggctatcggagctggcacctgggatatttcgctcgccacctccactggcaagttcgttgtcccccacctgcccgtggaggagtttatgttctttgctctgatcaacaccgtgctcgttttcggtacttgcgccatcgaccgaaccatggctattctgcatctctttaagaacaagtctccctaccagcgaccttaccagcactctaagtcgttcctgcatcagatcctcgagatgacttgggccttttgtctgcccgaccaggtgctccactccgacaccttccatgatctctccgtttcttgggatattctgcgaaaggcctcgaagtccttctacaccgcctctgctgtctttcccggtgacgtgcgacaggagctgggagtcctctacgccttctgccgagctaccgacgatctgtgtgacaacgagcaggtccctgtgcagactcgaaaggagcagctgatcctcacccaccagttcgtgtcggacctctttggtcagaagacctccgcccccactgctatcgactgggatttctacaacgaccagctgcctgcctcctgcatttctgctttcaagtcctttacccgactgcgacacgtcctcgaggctggagctatcaaggagctgctcgacggttacaagtgggatctcgagcgacgatctattcgagaccaggaggatctgcgatactactcggcctgcgtggcttcctctgttggagagatgtgtacccgaatcattctggctcacgctgacaagcctgcttcccgacagcagacccagtggatcattcagcgagctcgagagatgggtctggtcctccagtacactaacatcgcccgagacattgtgaccgattctgaggagctgggacgatgttacctccctcaggactggctgaccgagaaggaggtcgctctcatccagggaggtctggctcgagagattggagaggagcgactgctctctctgtcgcaccgactcatctaccaggccgacgagctcatggtggttgctaacaagggcattgataagctgccttcccattgccagggaggcgtgcgagccgcttgtaacgtttacgcctctatcggaaccaagctgaagtcgtacaagcaccattacccctcccgagcccacgtcggcaactctaagcgagtggagatcgctctgctctctgtgtacaacctctacaccgcccctattgctacttcgtccaccactcattgccgacagggcaagatgcgaaacctgaacaccatctaa

SEQ ID NO: 29 - protein, 614 aa, Artificial Sequence
  SYNTHESIZED
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 30 - DNA, 1845 bp, Artificial Sequence; identical to SEQ ID NO: 28
  SYNTHESIZED

SEQ ID NO: 31 - protein, 614 aa, Artificial Sequence
  SYNTHESIZED
MLLTYMEVHLYYTLPVLGVLSWLSRPYYTATDALKFKFLTLVAFTTASAWDNYIVYHKAWSYCPTCVTAVIGYVPLEKYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLSVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 32 - DNA, 2028 bp, Artificial Sequence
  SYNTHESIZED
atgtccaaggcgaaattcgaaagcgtgttcccccgaatctccgaggagctggtgcagctgctgcgagacgagggtctgccccaggatgccgtgcagtggttttccgactcacttcagtacaactgtgtgggtggaaagctcaaccgaggcctgtctgtggtcgacacctaccagctactgaccggcaagaaggagctcgatgacgaggagtactaccgactcgcgctgctcggctggctgattgagctgctgcaggcgtttttcctcgtgtcggacgacattatggatgagtccaagacccgacgaggccagccctgctggtacctcaagcccaaggtcggcatgattgccatcaacgatgctttcatgctagagagtggcatctacattctgcttaagaagcatttccgacaggagaagtactacattgaccttgtcgagctgttccacgacatttcgttcaagaccgagctgggccagctggtggatcttctgactgcccccgaggatgaggttgatctcaaccggttctctctggacaagcactcctttattgtgcgatacaagactgcttactactccttctacctgcccgttgttctagccatgtacgtggccggcattaccaaccccaaggacctgcagcaggccatggatgtgctgatccctctcggagagtacttccaggtccaggacgactaccttgacaactttggagaccccgagttcattggtaagatcggcaccgacatccaggacaacaagtgctcctggctcgttaacaaagcccttcagaaggccacccccgagcagcgacagatcctcgaggacaactacggcgtcaaggacaagtccaaggagctcgtcatcaagaaactgtatgatgacatgaagattgagcaggactaccttgactacgaggaggaggttgttggcgacatcaagaagaagatcgagcaggttgacgagagccgaggcttcaagaaggaggtgctcaacgctttcctcgccaagatttacaagcgacagaagggtggtggttctatggattataacagcgcggatttcaaggagatatggggcaaggccgccgacaccgcgctgctgggaccgtacaactacctcgccaacaaccggggccacaacatcagagaacacttgatcgcagcgttcggagcggttatcaaggtggacaagagcgatctcgagaccatttcgcacatcaccaagattttgcataactcgtcgctgcttgttgatgacgtggaagacaactcgatgctccgacgaggcctgccggcagcccattgtctgtttggagtcccccaaaccatcaactccgccaactacatgtactttgtggctctgcaggaggtgctcaagctcaagtcttatgatgccgtctccattttcaccgaggaaatgatcaacttgcatagaggtcagggtatggatctctactggagagaaacactcacttgcccctcggaagacgagtatctggagatggtggtgcacaagaccggtggactgtttcggctggctctgagacttatgctgtcggtggcatcgaaacaggaggaccatgaaaagatcaactttgatctcacacaccttaccgacacactgggagtcatttaccagattctggatgattacctcaacctgcagtccacggaattgaccgagaacaagggattctgcgaagatatcagcgaaggaaagttttcgtttccgctgattcacagcatacgcaccaacccggataaccacgagattctcaacattctcaaacagcgaacaagcgacgcttcactcaaaaagtacgccgtggactacatgagaacagaaaccaagagtttcgactactgcctcaagaggatacaggccatgtcactcaaggcaagttcgtacattgatgatctagcagcagctggccacgatgtctccaagctacgagccattttgcattattttgtgtccacctctgactgtgaggagagaaagtactttgaggatgcgcagtga

SEQ ID NO: 33 - protein, 675 aa, Artificial Sequence
  SYNTHESIZED
MSKAKFESVFPRISEELVQLLRDEGLPQDAVQWFSDSLQYNCVGGKLNRGLSVVDTYQLLTGKKELDDEEYYRLALLGWLIELLQAFFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGSMDYNSADFKEIWGKAADTALLGPYNYLANNRGHNIREHLIAAFGAVIKVDKSDLETISHITKILHNSSLLVDDVEDNSMLRRGLPAAHCLFGVPQTINSANYMYFVALQEVLKLKSYDAVSIFTEEMINLHRGQGMDLYWRETLTCPSEDEYLEMVVHKTGGLFRLALRLMLSVASKQEDHEKINFDLTHLTDTLGVIYQILDDYLNLQSTELTENKGFCEDISEGKFSFPLIHSIRTNPDNHEILNILKQRTSDASLKKYAVDYMRTETKSFDYCLKRIQAMSLKASSYIDDLAAAGHDVSKLRAILHYFVSTSDCEERKYFEDAQ

SEQ ID NO: 34 - DNA, 1953 bp, Marchantia polymorpha
atggtagaattatcaatcaacatgagttctagcttgagcctcgagagtgtgtgctccgccaggtgtttttcaccttcctcatcggccataggagcagtccccggggttcgtaggaaattatgtgtatccgtgagagagaagccggaacaacctgtgggcgcagttttcgtggggtgctcaacgaagcatcgaaagtcgagaaatcacgaaatgtggagtagcagcagggattgcatcacttcggctcattctgcaggtttggatttcgcgtcttcgaaggaaggcaatgcttgcgccacgacgtcgtcgaagtcgggtgcacgatttttgcacgatgagggaatgggaaccatagatcgggcagaagcggttcgagcgcaattgtttcctagattgaacaagttgtcccccgtcaaaagcctgcggcggagatgcgtttctccatccacacgggttgtcaccagcgtcctcgtcccacctcgtgaacaatatgccgacgagacggattatatgaaagctgggggagaatttatcgatctcgtgcagctacaagccagaaaacctctccagcaaacgaaaattggagaaaagttggagccactctcggataagcttttggatcttgtagtcatcggatgtggacctgccggtctgtcgctagcagctgaggctgcgaagcagggtttggaggttggcctcatcggccccgacttacctttcaccaacaattacggcgtgtgggaagacgaatttgcagcgttaggactggagaattgtatcgagcagatttggagagactctgcgatgtatttcgaaagtgataccccactgctgataggacgtgcctatggccgagtggatcgtcatctgctccatgaagagctgttgaaaagatgcgctgatggaggtgtacagtacctcgacactgaagtcgagaggatctccgacgcagacgacactgggagcacagttatgtgcgccaacggagctgtgatcagatgcagactggtcacagttgcctccggagcagccgcgggtcgttttttggagtatgagccaggtggtcctggaactacggttcagaccgcttatgggatggaagttgagtgcgagaacttcaattacgatcctgaaattatgctcttcatggattatcgggactatcaagcatggggaaccgaaccatgtccggatgccgatgagttcaaacaagtgccttcatttctctacgcaatgccagtttcgaaaactagagttttctttgaggagacttgcctggcagcaaggccgactatgtccttcaaccttttaaaggagagactgctcatgcgattaaactccatgggcattaaggtggtgcacatgtacgaggaggaatggtcctatattcctgtcggggcaacgctccctgatacgacgcagcaacatttgggcttcggggcagccgcgagcatggttcatcctgccaccggatattctgtggttcgatccctgtcggaggctcctcattacgctgcagccattgcctcctcactgcggtctggaggaaagagtgtggatgtaaattcgatggtaattcaaagttggaagcatcccagagctgcagctttagaagcttggaatgcactatggccgagcgagaggaagcgtcaacgagcatttttcttgttcgggcttgagctgattttgcaactcgatctcgtgggcatacgagaattcttcgccaccttcttcgaactacctgaatggctgtggaaagggtttctcgcagcgaaattgtcgtccctggacctgattatgttcgcactgattacgtttgtagtagcccccaactctctccgctaccgactggtaaggcacctgatgacggatcccagcggatcgtacttgattcgcacgtacttgggattgaaaggcactgcggagctgccggctgcgaaggagatgagatga

SEQ ID NO: 35 - DNA, 1611 bp, Artificial Sequence
  SYNTHESIZED
atgggcaccatcgaccgagctgaggctgtccgagctcagctgttcccccgactcaacaagctgtcccccgtcaagtctctccgacgacgatgcgtgtccccctctacccgagtcgtgacctctgtcctggtgcccccccgagagcagtacgctgacgagaccgactacatgaaggctggcggagagttcatcgacctcgtgcagctgcaggcccgaaagcccctgcagcagaccaagattggagagaagctcgagcccctgtctgacaagctgctcgacctcgtcgtgatcggatgtggacctgctggactgtccctcgctgctgaggctgctaagcagggactcgaggtcggactgattggtcccgacctgcccttcaccaacaactacggcgtgtgggaggacgagttcgccgctctgggtctcgagaactgcatcgagcagatttggcgagactccgccatgtacttcgagtctgacacccccctgctcatcggtcgagcttacggacgagtggaccgacacctgctccacgaggagctgctcaagcgatgtgccgacggaggcgtccagtacctggacaccgaggtggagcgaatctccgacgctgacgacaccggatctaccgtcatgtgcgccaacggcgctgtgattcgatgccgactggtcaccgtggcttccggagccgctgccggtcgattcctcgagtacgagcccggtggccccggcaccaccgtccagaccgcctacggaatggaggtggagtgcgagaacttcaactacgaccccgagattatgctgttcatggactaccgagactaccaggcttggggcaccgagccttgtcctgacgctgacgagttcaagcaggtgccctccttcctgtacgccatgcccgtctctaagacccgagtgttcttcgaggagacctgcctcgctgcccgacccaccatgtccttcaacctgctcaaggagcgactgctcatgcgactgaactctatgggcatcaaggtcgtgcacatgtacgaggaggagtggtcctacattcctgtcggtgctaccctgcctgacaccacccagcagcacctcggtttcggagctgccgcttccatggtgcaccctgctaccggttactctgtcgtgcgatccctgtctgaggctcctcactacgccgctgccatcgcttcctctctccgatccggcggcaagtctgtggacgtgaactccatggtcattcagtcttggaagcacccccgagctgccgctctggaggcttggaacgctctctggccctctgagcgaaagcgacagcgagccttcttcctgttcggtctggagctcatcctgcagctcgacctggtgggcatccgagagttcttcgctaccttcttcgagctccccgagtggctgtggaagggattcctcgccgctaagctgtcctctctcgacctgatcatgttcgccctgattaccttcgtcgtggctcccaactccctccgataccgactggtccgacacctcatgaccgacccctccggctcttacctgattcgaacctacctcggactgaagggcaccgctgagctccctgctgctaaggagatgcgataa

SEQ ID NO: 36 - protein, 536 aa, Artificial Sequence
  SYNTHESIZED
MGTIDRAEAVRAQLFPRLNKLSPVKSLRRRCVSPSTRVVTSVLVPPREQYADETDYMKAGGEFIDLVQLQARKPLQQTKIGEKLEPLSDKLLDLVVIGCGPAGLSLAAEAAKQGLEVGLIGPDLPFTNNYGVWEDEFAALGLENCIEQIWRDSAMYFESDTPLLIGRAYGRVDRHLLHEELLKRCADGGVQYLDTEVERISDADDTGSTVMCANGAVIRCRLVTVASGAAAGRFLEYEPGGPGTTVQTAYGMEVECENFNYDPEIMLFMDYRDYQAWGTEPCPDADEFKQVPSFLYAMPVSKTRVFFEETCLAARPTMSFNLLKERLLMRLNSMGIKVVHMYEEEWSYIPVGATLPDTTQQHLGFGAAASMVHPATGYSVVRSLSEAPHYAAAIASSLRSGGKSVDVNSMVIQSWKHPRAAALEAWNALWPSERKRQRAFFLFGLELILQLDLVGIREFFATFFELPEWLWKGFLAAKLSSLDLIMFALITFVVAPNSLRYRLVRHLMTDPSGSYLIRTYLGLKGTAELPAAKEMR

SEQ ID NO: 37 - DNA, 1773 bp, Chlamydomonas reinhardtii
atgatgcttaaagctggaaaccgacctgtggccctgcgctcgggccgcagcgcgactgtgtctccaatcagtcgcgttgtgtcccgtccccagcagctgcagaggcgcatatgcactgctgcagctggtcagaaggacgcatttccgtccgggccgtatcctattccgcctggccccgttgggcacttctaccgcgagaccgagaaatggcccacctctgagaccgttaggcttcagccgcatgacttaaacgaggtagactatgttgacctggtggtggctggcgcgggcccggctggtgtcgcggtggcctcccgcgtcgctgccgcgggcttctcagtttgcgttgtcgaccccgagccgctggcccactggcccaacaactatggtgtctggctcgatgagttccaggcgatggggctggaggactgcctgcacgtcatctggcccaaggccaaggtctggctcaacagcgaggccgacggcgagaagttcctgaaccgccccttcggccgcgtggaccggcccaagctgaagcgcatcctgctggagcgctgcgtcgcctcgggcgtgacgttccttgacgccaaggtgtcgggcgtgagtcacggcggcggctgcagcgccgtcaagctggcggacggccgcgagatccgcggcagcctggtcctggacgccaccggccactcgcgccgcctggtgcagtacgacaagaagttcgacccgggcttccagggcgcgtacggcattgtggcggaggttgagtctcacccgtttgcgctggacaccatgctgttcatggactggcgcgacgaccacacgcaggcgccggggctggaggccatgcgcgcagccaacaccgcgctgcccaccttcctgtacgccatgcccttcaccaagaacctggtgttcctggaggagaccagcctggtgtcgcggcccgcggtggacttccccgagctcaaggaccgcctgcaggcgcggctgcagcacctggggatcaaggtgaccaacgtgctggaggaggagtactgcctcatccccatgggcggcgtgctgcccaaacacccacagcgcgtgctggccattggcggcacagccggcatggtgcatccctccacgggcttcatgatcagccgcatgatgggcgcggcgcccacggttgccgacaccattgtggatcagctcagtcgccccgccgacaaggccagcgagtcaggcgccccgctgcgcccctccagcgaggcggaggcggagtccatggccgccgccgtgtgggccgccacctggccgctggagcgcgtgcggcagcgcgccttcttcacctttggcatggacgtgctgctcaagctcaacctgccgcagatccgggagttcttcagggcgttcttcagcctcagtgacttccactggcacggcttcctgtccacacgcctgtcgctgccgcagctgattgtgtttggcctgacgctgttctggaagagctccaaccaggctcgcgccagcctgctgcagctgggcatccccggcctggtggtgatgctgtcgggactggcgcccacactgggaggcggctactacccagacacaatgtcgctcaaggagcgcaaggacgcagtggacgccgccgcgcgctccgccgccgccgccgcccgcgccgccgcggacgtggccagcgacgccgccgccttcgtgtccgccaactcgagcggcgccgacatggcggtggtggaggtggtggagaaggcgttcagcaccagcaacaccaagtaa

SEQ ID NO: 38 - DNA, 1773 bp, Artificial Sequence
  SYNTHESIZED
atgatgctgaaggctggtaaccgacctgtggctctccgatctggccgatctgctactgtgtctcctatttcccgagtggtgtcccgaccccagcagctgcagcgacgaatctgtaccgccgctgccggtcagaaggacgcctttccctctggcccctaccctattccccctggccctgtcggacacttctaccgagagaccgagaagtggcccacctccgagactgttcgactgcagcctcatgacctcaacgaggttgactacgtggatctggtggtcgctggagctggtcctgctggagtggctgtcgcctcgcgagtcgctgccgctggtttttctgtttgtgttgtggaccccgagcctctcgctcactggcctaacaactacggcgtgtggctggacgagttccaggccatgggactggaggattgcctccatgtgatctggcctaaggctaaggtctggctcaactctgaggccgacggagagaagttcctgaaccgaccctttggtcgagtggatcgacctaagctgaagcgaattctgctcgagcgatgtgtcgcttccggtgttaccttcctggacgctaaggtctccggcgtttcgcacggtggaggttgctcggctgtcaagctggccgacggacgagagatccgaggttccctggttctcgatgccaccggacattcgcgacgactggtgcagtacgacaagaagttcgatcccggttttcagggcgcttacggaattgtggccgaggtcgagtcccacccttttgccctggacaccatgctcttcatggattggcgagacgatcatactcaggctcccggtctggaggctatgcgagctgctaacaccgctctgcccactttcctctacgccatgccttttaccaagaacctggtgttcctcgaggagacttctctggtgtcccgacccgctgtggactttcctgagctgaaggatcgactccaggcccgactgcagcacctcggaatcaaggttaccaacgtgctggaggaggagtactgcctcattcccatgggcggagttctgcccaagcaccctcagcgagtgctcgctatcggtggtaccgctggcatggtccatccttccactggattcatgatctcgcgaatgatgggtgccgctcccaccgttgctgacactattgtggatcagctctctcgacctgctgacaaggcttctgagtccggagcccccctgcgaccttcttccgaggctgaggctgagtccatggccgctgccgtgtgggctgctacctggcccctggagcgagtccgacagcgagccttctttactttcggcatggacgtgctgctcaagctgaacctccctcagattcgagagttctttcgagccttcttttcgctgtctgacttccactggcatggctttctctcgacccgactgtctctcccccagctgatcgtcttcggactgactctcttttggaagtcgtctaaccaggctcgagcctctctgctccagctgggtattcccggcctcgtcgttatgctgtccggactcgctcctaccctcggaggtggctactaccctgacactatgtcgctgaaggagcgaaaggacgctgtcgatgctgccgctcgatctgccgctgccgctgcccgagctgccgctgacgtcgcttctgatgccgctgccttcgtttccgccaactcctcgggcgctgatatggctgtggttgaggtggtggagaaggctttttcgacctctaacactaagtag

SEQ ID NO: 39 - protein, 590 aa, Artificial Sequence
  SYNTHESIZED
MMLKAGNRPVALRSGRSATVSPISRVVSRPQQLQRRICTAAAGQKDAFPSGPYPIPPGPVGHFYRETEKWPTSETVRLQPHDLNEVDYVDLVVAGAGPAGVAVASRVAAAGFSVCVVDPEPLAHWPNNYGVWLDEFQAMGLEDCLHVIWPKAKVWLNSEADGEKFLNRPFGRVDRPKLKRILLERCVASGVTFLDAKVSGVSHGGGCSAVKLADGREIRGSLVLDATGHSRRLVQYDKKFDPGFQGAYGIVAEVESHPFALDTMLFMDWRDDHTQAPGLEAMRAANTALPTFLYAMPFTKNLVFLEETSLVSRPAVDFPELKDRLQARLQHLGIKVTNVLEEEYCLIPMGGVLPKHPQRVLAIGGTAGMVHPSTGFMISRMMGAAPTVADTIVDQLSRPADKASESGAPLRPSSEAEAESMAAAVWAATWPLERVRQRAFFTFGMDVLLKLNLPQIREFFRAFFSLSDFHWHGFLSTRLSLPQLIVFGLTLFWKSSNQARASLLQLGIPGLVVMLSGLAPTLGGGYYPDTMSLKERKDAVDAAARSAAAAARAAADVASDAAAFVSANSSGADMAVVEVVEKAFSTSNTK

SEQ ID NO: 40 - DNA, 1641 bp, Chromochloris zofingiensis
atggagtcaaaactgctgcgcaatactggcacattaggtgcaacgagacaactagtgcatgcatcctgcacttatcattatagaacagctgtgccaggttcacaaggtggcactttctgtgttcgtcatccgcggctgccactcaaggttcaggctgcagctacccttgaaagacccagcacatctggcaagtcacaattctacgttcgcgacccagccccatggccaactgacgttccaatccagcagcacgatcccaagaaaacacctttcgtggatttggtagtggcaggggccgggccatctgggctagctgttgctgaacgggtggcgcgcgcaggcttcacagtctgcatcatagacccaaatgcacttggagtctggcccaacaactacggcgtctgggtggatgagtttcaagcaatgggcttggatgactgtttggaggtcatttggccaaaagcaaaagtgtggctgaacaatagcaacgcaggcgaaaaattcctgtcaagaccatatggtcgagtggacaggcccaagcttaaacggaagttactggagagatgtgcagccagcggtgtgacattccttactggcaaggtggagggtgtaagacatggtgatggctcatcaacagtcagcacagcagagggtgtcagcctacaagggtcattggtgttggatgcaactggccacacgcgcaagcttgtgcagtttgacaagaagtttgatcctgggtatcagggcgcatatggcatcatagcagaggtcgagtcccatccgtttgaggttgacaccatgctgttcatggactggagggatgagcacttggccagccagccagacatgcgtgaacgcaacagtaagcttcccaccttcttatatgccatgccattcagcaagaccaagatctttctggaagagacgtccctggtcgcccggccagcagtgggattccaagatctgaaagacaggctagaagcacgcatgaagtggctaggaatcaaggtcaaacacattgaggaggaggagtactgcttgatccccatgggtggggtactgcccaagcacccccagcgtgtgttgggtattggtggcacagcaggcatggtgcatccctctactggcttcatggtatcacggatgctgggggtagcacccaccattgcagatgccatcattgaccagttgtccaaaccagcagacagggctgcagactcagctgtcgctctacgtccacagtctgagactgaagcaaacaatatggcggcagctgtgtggcgaacagcgtggccggtagagcggcttaggcagcgtgcattcttctgttttggcatggatgtgctgctaaggctggatttgcagcagaccagggagttcttcacagcattcttcagcctttcagacttccactggcacggcttcttatcagccagactatcattcccgcagcttatagggtttggtctcagcctgttcacaaaatccagcaaccaagcacgcatcaatctgttagccatgggcctacctggcctgctatcaatgcttgctgggctagcccctaccctaggccagtactacaagatcccagatggtgagctcggcagccttagtaaggcaagggcacaggtgaagagctag

SEQ ID NO: 41 - DNA, 1641 bp, Artificial Sequence
  SYNTHESIZED
atggagtctaagctgctgcgaaacaccggaaccctgggagccacccgacagctcgtccacgcctcttgcacctaccactaccgaaccgctgtgcccggctctcagggtggaaccttctgtgtccgacaccctcgactgcccctcaaggttcaggctgctgctaccctggagcgaccctccacttcgggcaagtcgcagttttacgtgcgagaccccgctccttggcctaccgatgtccctatccagcagcatgaccctaagaagactcctttcgtggacctggtggtcgctggtgctggaccttctggtctcgctgtggctgagcgagtcgcccgagctggcttcaccgtttgtatcattgatcctaacgccctgggcgtgtggcccaacaactacggagtttgggtggacgagttccaggctatgggactggacgattgcctcgaggttatttggcccaaggccaaggtctggctgaacaactccaacgctggagagaagtttctctcgcgaccttacggtcgagtggaccgacccaagctgaagcgaaagctgctcgagcgatgcgctgcctctggagtcaccttcctgactggaaaggtcgagggtgttcgacacggtgacggctcttccaccgtctccactgccgagggcgtttccctccagggatcgctggtcctcgacgctaccggccatactcgaaagctggttcagttcgacaagaagtttgatcccggctaccagggagcctacggtatcattgctgaggtcgagtctcacccttttgaggttgataccatgctgttcatggactggcgagatgagcatctcgcctcgcagcctgacatgcgagagcgaaactctaagctgcctactttcctctacgctatgcccttttccaagaccaagatcttcctggaggagacttcgctcgttgcccgacccgctgtgggtttccaggacctgaaggatcgactcgaggcccgaatgaagtggctgggcatcaaggtgaagcacattgaggaggaggagtactgtctgatccctatgggtggcgttctccctaagcacccccagcgagtgctgggtattggaggtaccgccggcatggtccatccctcgactggtttcatggtgtctcgaatgctgggtgtcgctcctaccattgctgacgctatcattgatcagctctccaagcccgctgaccgagctgccgattctgccgtggctctgcgacctcagtccgagaccgaggccaacaacatggctgctgctgtgtggcgaactgcttggcctgtcgagcgactgcgacagcgagctttcttttgctttggaatggacgtcctgctccgactggatctccagcagacccgagagttctttactgccttcttttctctgtccgacttccactggcatggttttctgtctgctcgactctccttccctcagctgatcggattcggtctgtcgctctttaccaagtcgtctaaccaggcccgaattaacctgctcgctatgggcctccccggactgctctctatgctggccggactcgctcctaccctgggacagtactacaagatccccgacggagagctgggctcgctctctaaggcccgagctcaggtgaagtcctag

SEQ ID NO: 42 - protein, 546 aa, Artificial Sequence
  SYNTHESIZED
MESKLLRNTGTLGATRQLVHASCTYHYRTAVPGSQGGTFCVRHPRLPLKVQAAATLERPSTSGKSQFYVRDPAPWPTDVPIQQHDPKKTPFVDLVVAGAGPSGLAVAERVARAGFTVCIIDPNALGVWPNNYGVWVDEFQAMGLDDCLEVIWPKAKVWLNNSNAGEKFLSRPYGRVDRPKLKRKLLERCAASGVTFLTGKVEGVRHGDGSSTVSTAEGVSLQGSLVLDATGHTRKLVQFDKKFDPGYQGAYGIIAEVESHPFEVDTMLFMDWRDEHLASQPDMRERNSKLPTFLYAMPFSKTKIFLEETSLVARPAVGFQDLKDRLEARMKWLGIKVKHIEEEEYCLIPMGGVLPKHPQRVLGIGGTAGMVHPSTGFMVSRMLGVAPTIADAIIDQLSKPADRAADSAVALRPQSETEANNMAAAVWRTAWPVERLRQRAFFCFGMDVLLRLDLQQTREFFTAFFSLSDFHWHGFLSARLSFPQLIGFGLSLFTKSSNQARINLLAMGLPGLLSMLAGLAPTLGQYYKIPDGELGSLSKARAQVKS

SEQ ID NO: 43 - DNA, 1023 bp, Marchantia polymorpha
atgctgaaggtcgtagcatctggcgccaccgctgttgcctctctcggggtcgtgaggagcggtcgtgaatgtgggcgagatgggattgggctcgagcagctgagacacagggcgctgccgagcttcccgagtctgggattgagcagcttggaatttaatccgttgatgacgagaaccggaattcaacgcaggattcgaattcagcgcagcatcggtcctccctccgttttgcagattgatgagcaccagcatggtgaatctccagctcccatcgaggagcacctgctcgagactgagcaatccgctgatgttgccgacaaagttgagagcagttttcccgacactcccgctgtcagtaaaatgcagcaaagattgaccaagagacaaacagaacgtaaagcatacctcttagcagccatcgcatctacgacaggattcaccacgctcgccgtcgccgccgtcttctatcgttttatctggcaaatgcagggaagtggcgagatcccgtacacagaaatattcggaacatttgccctcgctgtcggagccgcggttgggatggaatactgggctagatgggcgcacaaagctctgtggcacgcatcgctgtggaacatgcacgagtcacatcaccgacccagagaaggaccttttgaaatgaacgacatttttgctatcataaacgcagttcccgccgtctctctgatgctctacggatttcttaacagaggacttgttcctggtctctgcttcggagcgggtttaggcatcactatgttcggtatagcctacatgttcgttcacgatggtcttgtacaccgacgattcccagtcggacctatcgccgatgtcccatatcttcagaaggttgccgcagcccatcagcttcatcacgctgacctttacgagggtgtaccctatggtcttttcctcggcccaaaggagctggaagaagttggaggattggacgaactcgagagagtcatgaagcagagagccaagccctctgcatcctccaagtag

SEQ ID NO: 44 - DNA, 663 bp, Artificial Sequence
  SYNTHESIZED
atgcagcagcgactgaccaagcgacagaccgagcgaaaggcctacctgctcgccgctatcgcttccaccaccggtttcaccaccctcgctgtcgctgctgtgttctaccgattcatttggcagatgcagggatctggcgagatcccctacaccgagattttcggaaccttcgctctggctgtcggtgctgctgtgggaatggagtactgggctcgatgggctcacaaggctctctggcacgcttccctgtggaacatgcacgagtctcaccaccgaccccgagagggacccttcgagatgaacgacatcttcgccatcattaacgccgtccccgctgtgtccctgatgctctacggcttcctgaaccgaggactcgtgcccggtctgtgcttcggtgctggactcggcatcaccatgttcggaattgcttacatgttcgtccacgacggactggtgcaccgacgattccctgtcggtcccattgccgacgtgccctacctccagaaggtcgccgctgcccaccagctccaccacgctgacctgtacgagggagtcccctacggcctgttcctcggtcctaaggagctggaggaagtgggaggtctggacgagctcgagcgagtcatgaagcagcgagccaagccctctgcttcctctaagtaa

SEQ ID NO: 45 - protein, 220 aa, Artificial Sequence
  SYNTHESIZED
MQQRLTKRQTERKAYLLAAIASTTGFTTLAVAAVFYRFIWQMQGSGEIPYTEIFGTFALAVGAAVGMEYWARWAHKALWHASLWNMHESHHRPREGPFEMNDIFAIINAVPAVSLMLYGFLNRGLVPGLCFGAGLGITMFGIAYMFVHDGLVHRRFPVGPIADVPYLQKVAAAHQLHHADLYEGVPYGLFLGPKELEEVGGLDELERVMKQRAKPSASSK

SEQ ID NO: 46 - DNA, 1779 bp, Marchantia polymorpha
atggctgcatccatggcgcaaatgctgcccgtgcaattctcctctagacgctcactgggtccttcttcctccgctagaaggtgtgggaaggtggcaaattctctgcaatgcagcagaatttgtgggcttcggaatgtcggattctctagctctctaccgagtccgagacaggatttcaatagaatggagtgcaatgggtatggggccgcgtccaggtttgcaagacaggtgattcgctcggatatggagaaagagagtggcaaagtgctgaataaacagggggcgggaaaatcgtgggtcagcccggactggctaactggtttggtacagatggtgaaggggaaggatgaatcaggtattcctatagctgacgcaaaattggaggatgtgcaggaccttctgggcggagctctgttcttgcctttgttcaagtggatgaaagagtcgggcccaatttacaggttggcagcaggaccgaggaatttcgtgattgtcagtgatccccagatggcgaagcatgtgctgcgagcttatggaacaaagtacgcgaaggggctcgtagcagaggtggctgagtttttattcggttcgggatttgccattgcagagaaccagctctggactgttcgcaggagagcggtagtcccatctctccatcgaaagtatctggcgactatggtggatcgcgtgttttgtagatgtgcggagagactcgtggacacattacaggctgctgacgagaaaggtgtagctgtgaacatggaagcaagattctctcagctgaccctggatgttatcgggttgtccgtcttcaactacgacttcgattctcttacatcagatagtccggtcatagaagctgtttacacggctttgaaggaaacagagtcaagatccacagacatactaccatactggcaggtgccttttttgtgccaaatagtacccaggcaacagaaagctgcaaaggcagtcgcgcttattcgagaaactgtcgaagacctggtagcgaagtgtaagaaaattgtggatgaagaaggggagagattggagggtgaagaatatatcaacgaagcagatccctcagtgcttcgtttcctcctggcaagtcgtgaagaggtctcgagcacccagttacgggatgacctcctttctatgcttgtagcagggcacgagaccacaggctccgtcctcacctggaccgtctatttgttaagcaagaatccttcagcttaccaaaaaatgcaagaagaactcgatacagttttggggggtagaaatcccacaatggaggacgttaagaacttgaagtacctaactcggtgtattaacgagtctatgcgattgtatcctcatccaccggtattgatcagaagagccaatgcgccagatacgttgcctggaggttacaaactcggagctggacaagacgttatgatatctgtttataatattcaccactcacctgctgtgtgggaaagagcagaagagttcatccctgagaggtttgatctggagggtcctgtccctaatgagagcaacacagattacagatacatacccttcagcggaggtcctcgtaagtgcgttggggaccaatttgccatgctggaagcgattgtcgctctggccgtggttcttcaacgcttccacttttcgcttgtccccaaccagactatagggatgacaactggagccaccatccacaccacctcgggactcttcatgaatgtgaaggctaggcaaaagaaaccagcagctgagctcgcaagcatataa

SEQ ID NO: 47 - DNA, 1545 bp, Artificial Sequence
  SYNTHESIZED
atgtccgacatggagaaggagtctggcaaggtcctcaacaagcagggagccggcaagtcctgggtgtctcccgactggctcaccggactggtccagatggtgaagggcaaggacgagtctggaatccccattgccgacgctaagctggaggacgtccaggacctgctcggcggtgctctgttcctccccctgttcaagtggatgaaggagtccggacctatctaccgactcgctgctggtccccgaaacttcgtcattgtgtctgacccccagatggccaagcacgtgctgcgagcttacggcaccaagtacgccaagggactcgtcgccgaggtggctgagttcctgttcggttctggattcgccatcgctgagaaccagctgtggaccgtccgacgacgagccgtcgtgccctccctccaccgaaagtacctggccaccatggtggaccgagtgttctgccgatgtgctgagcgactcgtggacaccctgcaggccgctgacgagaagggagtcgccgtgaacatggaggctcgattctcccagctcaccctggacgtcattggcctctccgtgttcaactacgacttcgactctctgacctccgactctcccgtcatcgaggccgtgtacaccgctctgaaggagaccgagtcccgatctaccgacatcctcccctactggcaggtccccttcctgtgccagattgtgccccgacagcagaaggccgctaaggccgtcgctctcatccgagagaccgtcgaggacctggtggccaagtgtaagaagattgtggacgaggagggcgagcgactcgagggagaggagtacatcaacgaggctgacccctccgtcctgcgattcctgctcgcctctcgagaggaggtgtcctctacccagctccgagacgacctgctctccatgctggtcgctggacacgagaccaccggctctgtcctgacctggaccgtgtacctgctctccaagaacccctctgcttaccagaagatgcaggaggagctcgacaccgtcctgggaggccgaaaccccaccatggaggacgtgaagaacctcaagtacctgacccgatgcattaacgagtctatgcgactctaccctcaccctcctgtcctgatccgacgagccaacgctcctgacaccctccccggtggatacaagctgggagctggtcaggacgtcatgatctccgtgtacaacattcaccactctcccgccgtctgggagcgagctgaggagttcattcctgagcgattcgacctggagggacccgtgcccaacgagtccaacaccgactaccgatacattcccttctctggcggtccccgaaagtgtgtcggtgaccagttcgccatgctcgaggctatcgtggccctcgctgtcgtgctgcagcgattccacttctccctggtccccaaccagaccatcggaatgaccaccggcgccaccattcacaccacctctggcctcttcatgaacgtgaaggctcgacagaagaagcccgccgctgagctggcctccatctaa

SEQ ID NO: 48 - protein, 514 aa, Artificial Sequence
  SYNTHESIZED
MSDMEKESGKVLNKQGAGKSWVSPDWLTGLVQMVKGKDESGIPIADAKLEDVQDLLGGALFLPLFKWMKESGPIYRLAAGPRNFVIVSDPQMAKHVLRAYGTKYAKGLVAEVAEFLFGSGFAIAENQLWTVRRRAVVPSLHRKYLATMVDRVFCRCAERLVDTLQAADEKGVAVNMEARFSQLTLDVIGLSVFNYDFDSLTSDSPVIEAVYTALKETESRSTDILPYWQVPFLCQIVPRQQKAAKAVALIRETVEDLVAKCKKIVDEEGERLEGEEYINEADPSVLRFLLASREEVSSTQLRDDLLSMLVAGHETTGSVLTWTVYLLSKNPSAYQKMQEELDTVLGGRNPTMEDVKNLKYLTRCINESMRLYPHPPVLIRRANAPDTLPGGYKLGAGQDVMISVYNIHHSPAVWERAEEFIPERFDLEGPVPNESNTDYRYIPFSGGPRKCVGDQFAMLEAIVALAVVLQRFHFSLVPNQTIGMTTGATIHTTSGLFMNVKARQKKPAAELASI

SEQ ID NO: 49 - DNA, 1005 bp, Glycine max
atgggggataggggatcatcacattccttactcgcaggcgaacacaaacactctctctttgcctcttggcgcaattcgatcgaagctatctacccttccatggcggcaggactccccaccgccgcaatcttaaagccctacaatctcgtccaacccccaatccctctttctaaaccaaccacatcactcttcttcaaccccttaagatgtttccatcacagtacaatccttcgagttcgacccagaagaagaatgagcggcttcaccgtttgcgtcctcacggaggattccaaagagatcaaaacggtcgaacaagaacaagaacaagtgattcctcaagccgtgtcagcaggtgtggcagagaagttggcgagaaagaagtcccagaggttcacttatctcgttgcggctgtcatgtctagctttggcatcacctctatggcagtctttgccgtttattatagattctcctggcaaatggagggtggagatgttccttggtctgaaatgctaggcacattttccctctccgtcggtgctgctgtggctatggaattttgggcaagatgggctcatagagctctttggcatgcttccttgtggcacatgcacgagtcacaccatcgaccaagagagggaccgttcgagctcaacgacgttttcgcgataattaacgctgtccctgcgatcgctcttctctcatacggtattttccacaagggtctggtccctgggctctgttttggtgcaggccttggaatcacggtatttgggatggcctacatgtttgtccacgatggattagttcataagagattccctgtgggtcccattgccaacgtgccctacttcagaagagttgctgctgctcaccaactccaccattcggataaattcaacggggcgccatatggcctctttttgggaccaaaggaagttgaagaagtgggagggctagaagagctagagaaagagataagtaggagaatcaggtccggttcatga

SEQ ID NO: 50 - DNA, 1005 bp, Artificial Sequence
  SYNTHESIZED
atgggtgaccgaggctcctctcactctctcctggctggtgaacacaagcactccctgttcgcttcttggcgaaactccatcgaggccatttacccctctatggctgctggtctgcctaccgctgctatcctgaagccctacaacctcgtgcagcctcccattcccctctctaagcccaccacctccctgttcttcaaccccctccgatgcttccaccactccaccatcctgcgagtccgaccccgacgacgaatgtctggtttcaccgtctgtgtgctcaccgaggactccaaggagatcaagaccgtggagcaggagcaggagcaggtcattcctcaggctgtgtctgctggagtggctgagaagctggctcgaaagaagtctcagcgattcacctacctcgtcgccgctgtgatgtcctctttcggcattacctctatggccgtcttcgctgtgtactaccgattctcctggcagatggagggcggtgacgtgccctggtctgagatgctgggaaccttctccctctctgtcggcgccgctgtggctatggagttctgggctcgatgggctcaccgagctctgtggcacgcttctctctggcacatgcacgagtctcaccaccgaccccgagagggtcctttcgagctgaacgacgtgttcgctatcattaacgccgtccccgctatcgccctgctctcttacggaattttccacaagggcctggtgcccggtctctgcttcggtgctggactgggcatcaccgtcttcggtatggcctacatgttcgtccacgacggactcgtgcacaagcgattccccgtcggccccattgctaacgtgccctacttccgacgagtggccgctgcccaccagctgcaccactccgacaagttcaacggagccccctacggactgttcctcggtcccaaggaagtcgaggaagtcggcggcctggaggagctcgagaaggagatttcccgacgaatccgatccggatcttga

SEQ ID NO: 51 - protein, 334 aa, Artificial Sequence
  SYNTHESIZED
MGDRGSSHSLLAGEHKHSLFASWRNSIEAIYPSMAAGLPTAAILKPYNLVQPPIPLSKPTTSLFFNPLRCFHHSTILRVRPRRRMSGFTVCVLTEDSKEIKTVEQEQEQVIPQAVSAGVAEKLARKKSQRFTYLVAAVMSSFGITSMAVFAVYYRFSWQMEGGDVPWSEMLGTFSLSVGAAVAMEFWARWAHRALWHASLWHMHESHHRPREGPFELNDVFAIINAVPAIALLSYGIFHKGLVPGLCFGAGLGITVFGMAYMFVHDGLVHKRFPVGPIANVPYFRRVAAAHQLHHSDKFNGAPYGLFLGPKEVEEVGGLEELEKEISRRIRSGS

(19) United States
(12) Patent Application Publication
(10) Pub. No.: US 2018/0105839 A1
(43) Pub. Date: Apr. 19, 2018

(54) COMPOSITIONS AND METHODS OF BIOSYNTHESIZING XANTHOPHYLLS

(71) Applicant: ARCH INNOTEK, LLC, St. Louis, MO (US)

(72) Inventor: Yechun Wang, St. Louis, MO (US)

(21) Appl. No.: 15/694,443

(22) Filed: Sep. 1, 2017

Related U.S. Application Data
(60) Provisional application No. 62/409,599, filed on Oct. 18, 2016.

(51) Int. Cl. C12P 7/02; C12N 9/90; C12N 9/02; C12N 9/10

(52) U.S. Cl. CPC C12P 7/02; C12N 9/90; C12Y 505/01018; C12N 9/0071; C12Y 114/13129; C12Y 103/99; C12Y 114/99045; C12N 9/1085; C12Y 205/01032; C12N 9/001; C12N 9/0083

(57) ABSTRACT

The present invention relates to compositions and methods of producing xanthophylls in microorganisms.

### CROSS-REFERENCE TO RELATED APPLICATIONS

This application relates to and claims the priority of U.S. Provisional Patent Application Ser. No. 62/409,599, which was filed Oct. 18, 2016, and is hereby incorporated by reference in its entirety.

### FIELD OF THE INVENTION

This disclosure relates generally to a method for the biosynthetic production of xanthophylls by microorganisms, especially lutein and β-cryptoxanthin.

### BACKGROUND OF THE INVENTION

Carotenoids are a class of naturally occurring pigments with a 40-carbon backbone and a large conjugated double-bond system. Carotenoids are red, yellow and orange pigments that are widely distributed in nature. Among more than 700 carotenoids have identified thus far, as many as 50 may be absorbed and metabolized by the human body. The most abundant six carotenoids in human serum are α-carotene, β-carotene, β-cryptoxanthin, lycopene, lutein, and zeaxanthin.

There are two general classes of carotenoids: carotenes and xanthophylls. Carotenes typically consist only of carbon and hydrogen atoms such α-carotene, beta-carotene and lycopene. Xanthophylls have one or more oxygen atoms, and include compounds such as lutein, zeaxanthin and β-cryptoxanthin.

Lutein ((3R,3′R,6′R)-α, ε-carotene-3,3′-diol) is an antioxidant that has gathered increasing attention due to its potential role in preventing or ameliorating age-related macular degeneration (AMD). High levels of lutein in serum have been inversely correlated with lung cancer. Lutein occurs in maize, orange pepper, kiwi fruit, grapes, spinach, orange juice, zucchini, squash, red cabbage, broccoli and kale etc. Lutein is largely consumed as a food colorant and global lutein market has grown significantly in the recent years. The lutein market is segmented into pharmaceutical, nutraceutical, food, pet foods, and animal and fish feed. The pharmaceutical market is estimated to be about $190 million, nutraceutical and food is estimated to be about $110 million, pet foods and other applications are estimated at $175 million annually. In the EU, lutein is listed as E161b when used as feed additive. Currently, commercial sources are obtained from the extraction of marigold petals. However, marigold presents several drawbacks as a source of lutein. The flowers must be periodically harvested and petals separated prior to extraction. The lutein content in marigold petals is variable and can be as low as 0.03%. Lutein is present in plants as fatty-acid esters with one or two fatty acids bound to the two hydroxyl-groups. Saponification of lutein esters to yield free lutein may yield lutein in any ration from 1:1 to 1:2 molar ratios. In addition, the production of lutein from marigold is also limited by seasons, planting area, and the high cost of labor. Several microalgae have been considered as potential sources of lutein because they are capable of accumulating a much higher content (0.5%-1.2% dry weight) than marigold petals, and their growth is independent of season or weather. However, the disadvantage is the very low cell densities and long cultivation periods. Synthetic production of lutein is very inefficient and has a poor yield, at prices that cannot compete with marigold extraction. Compared with lutein production from plant materials, lutein production via microbial fermentation has a number of advantages including (1) cheaper production; (2) potentially increased ease of extraction; (3) free lutein form without further saponification needed; (4) higher yields (especially through strain improvement); (5) no lack of raw materials and (5) no seasonal variations.

β-cryptoxanthin ((3R)-beta,beta-caroten-3-ol) is a provitamin A carotenoid that has received attention in its role in human biological functions. Because of the free radical quenching ability and effects on cell differentiation and proliferation, multiple studies have suggested that β-cryptoxanthin protects against certain diseases such as cardiovascular disease, osteoporosis, and cancer. In addition, β-cryptoxanthin acts as an antioxidant in the body. Unlike the hydrocarbons or the dihydroxy-xanthophylls, β-cryptoxanthin has a bipolar structure due to its electronegative hydroxyl group on one side of the molecule and an unsubstituted β-ring on the other side, which yield vitamin A upon central cleavage. This unique bipolar nature allows β-cryptoxanthin to be easily deposited into the egg, hence not only enhancing the color of the egg yolk but also increasing the egg's vitamin A value. β-cryptoxanthin is also used as a substance to color food products (INS number 161c), it is approved for use in Australia and New Zealand.

Due to increasing interest in health benefits, there are several approaches to commercially produce β-cryptoxanthin. First, extract from natural sources; Second, biotechnology routes; Third, chemical synthesis. Foods that are rich in β-cryptoxanthin include papaya, mango, peaches, oranges, tangerines, corn and watermelon. However, unlike other carotenoids, β-cryptoxanthin is not found in most fruits or vegetables. No microorganism is capable to naturally producing β-cryptoxanthin. In 2008, a method is disclosed for preparing β-cryptoxanthin from a microorganism transformed with a truncated β-carotene hydroxylase from Arabidopsis thaliana (US2008/0124755). In 2009, a novel lycopene beta-monocyclase gene was used to transform a host cell and convert lycopene to β-cryptoxanthin through γ-carotene and 3-hydroxyl-γ-carotene (US2009/0093015 A1).

The chemical synthesis of β-cryptoxanthin for industrial production is not a very efficient or economically viable process. Such as, Khachik et al employed lutein as the staring material to produce α- and β-cryptoxanthin (US7115786B2). Although some methods have been reported, these elaborate synthetic methods are expensive and difficult to implement.

Therefore, there is a need for improved biological systems capable of efficiently providing natural, non-synthetic alternatives for xanthophylls, and in particular lutein and β-cryptoxanthin, at a lower cost.

### SUMMARY OF THE INVENTION

In one aspect, the present disclosure provides a recombinant microorganism comprising at least one artificial nucleic acid construct comprising a nucleic acid comprising a sequence encoding a lycopene ε-cyclase enzyme from Marchantia polymorpha, and a nucleic acid comprising a sequence encoding a lycopene β-cyclase enzyme. The lycopene β-cyclase enzyme may be selected from a lycopene β-cyclase enzyme from Chlamydomonas reinhardtii, a lycopene β-cyclase enzyme from Chromochloris zofingiensis, and a combination thereof. The nucleic acid sequences are operably linked to one or more expression control sequences. The microorganism may comprise α-carotene.

The nucleic acid expression construct comprising a nucleic acid sequence encoding lycopene ε-cyclase enzyme may comprise an amino acid sequence with at least 80% identity to an amino acid sequence of SEQ ID NO: 35. Additionally, the nucleic acid expression construct comprising a nucleic acid sequence encoding lycopene β-cyclase enzyme may comprise an amino acid sequence with at least 80% identity to an amino acid sequence selected from SEQ ID NO: 37 and SEQ ID NO: 41

The recombinant microorganism may further comprise a nucleic acid comprising a sequence encoding a β-carotene hydroxylase, and a nucleic acid comprising a sequence encoding a P450 carotene ε-ring hydroxylase, wherein the nucleic acid sequences are operably linked to one or more expression control sequences. The β-carotene hydroxylase enzyme may comprise the β-carotene hydroxylase enzyme from Marchantia polymorpha, and the P450 carotene ε-ring hydroxylase enzyme may comprise the P450 carotene ε-ring hydroxylase enzyme from Marchantia polymorpha. The microorganism may also further comprise a nucleic acid comprising a sequence encoding a phytoene synthase enzyme, and a nucleic acid comprising a sequence encoding a phytoene dehydrogenase enzyme, wherein the nucleic acid sequences are operably linked to one or more expression control sequences. The phytoene synthase enzyme may comprise a lycopene cyclase/phytoene synthase enzyme modified to decrease lycopene cyclase activity, wherein the enzyme is selected the group consisting Mucor circinelloides, Phycomyces blakesleeanus, and Xanthophyllomyces dendrorhous. The phytoene dehydrogenase enzyme may be selected from a phytoene dehydrogenase from Mucor circinelloides, from Xanthophyllomyces dendrorhous, and from Phycomyces blakesleeanus. The microorganism may comprise lutein.

The nucleic acid expression construct may comprise a nucleic acid sequence encoding β-carotene hydroxylase enzyme with at least 80% identity to an amino acid sequence of SEQ ID NO: 44. The nucleic acid expression construct may also comprise a nucleic acid sequence encoding P450 carotene ε-ring hydroxylase enzyme with at least 80% identity to an amino acid sequence SEQ ID NO: 47.

Any of the microorganisms disclosed above may be Yarrowia lipolytica or Saccharomyces cerevisiae. Additionally, any of the microorganisms disclosed above may further comprises nucleic acid sequences for producing geranyl geranyl diphosphate.

In another aspect, the present disclosure provides a method of producing lutein. The method comprises providing the recombinant microorganism disclosed above, cultivating the recombinant microorganism under conditions sufficient for the production of lutein, and isolating lutein from the recombinant microorganism.

In one aspect, the present disclosure provides a recombinant microorganism comprising at least one artificial nucleic acid construct comprising a nucleic acid having a sequence encoding a lycopene cyclase enzyme, and a nucleic acid having a sequence encoding a β-carotene hydroxylase enzyme from Glycine max. The nucleic acid sequences are operably linked to one or more expression control sequences. The lycopene cyclase enzyme and the phytoene synthase enzyme may comprise phytoene synthase and lycopene cyclase of a lycopene cyclase/phytoene synthase from Mucor circinelloides, and the phytoene dehydrogenase activity may comprise phytoene dehydrogenase from Mucor circinelloides.

The nucleic acid expression construct may comprise a nucleic acid sequence encoding β-carotene hydroxylase enzyme comprising an amino acid sequence with at least 80% identity to an amino acid sequence SEQ ID NO: 50.

The microorganism may further comprise a nucleic acid having a sequence encoding a phytoene synthase enzyme, and a nucleic acid having a sequence encoding a phytoene dehydrogenase activity, wherein the nucleic acid sequences are operably linked to one or more expression control sequences.

Any of the microorganisms disclosed above may be Yarrowia lipolytica or Saccharomyces cerevisiae. Additionally, any of the microorganisms disclosed above may further comprises nucleic acid sequences for producing geranyl geranyl diphosphate.

The microorganism may comprise β-cryptoxanthin.

In another aspect, the present disclosure provides a method of producing β-cryptoxanthin. The method comprises providing a recombinant microorganism disclosed above, cultivating the recombinant microorganism under conditions sufficient for the production of β-cryptoxanthin, and isolating β-cryptoxanthin from the recombinant microorganism.

In yet another aspect, the present disclosure provides an artificial nucleic acid expression construct for use in production of a xanthophyll, the nucleic acid encoding a polypeptide comprising an amino acid sequence with at least 80% identity to an amino acid sequence selected from SEQ ID NO: 35, SEQ ID NO: 37 and SEQ ID NO: 41, SEQ ID NO: 44, SEQ ID NO: 47, and SEQ ID NO: 50.

### BRIEF DESCRIPTION OF THE FIGURES

FIG. 1. Pathway for synthesis of lutein from GGPP in yeast. GGPP, geranylgeranyl diphosphate; carRP*, mutated phytoene synthase/lycopene cyclase; carB, phytoene dehydrogenase; LCYe, lycopene ε-cyclase; LCYb, lycopene β-cyclase; BHY, β-carotene hydroxylase; CYP97C, cytochrome P450 carotene ε-ring hydroxylase; BCH, β-carotene hydroxylase.

FIG. 2. Pathway for synthesis of β-Cryptoxanthin from GGPP in yeast. GGPP, geranylgeranyl diphosphate; carRP, biofunctional phytoene synthase/lycopene cyclase; carB, Phytoene dehydrogenase; BCH, β-carotene hydroxylase.

FIG. 3A depicts HPLC profiles of extracts from Y. lipolytica with exogenous expression of lycopene biosynthetic pathway genes (carRP*, carB and FPPS::GGPPS) showing generation of putative lycopene. FIG. 3B depicts HPLC profiles of extracts from Y. lipolytica with exogenous expression of lycopene biosynthetic pathway genes (carRP*, carB and FPPS::GGPPS) showing generation of authentic lycopene. FIG. 3C depicts UV spectra of putative lycopene peak at 29.33 min. FIG. 3D depicts UV spectra of authentic lycopene peak at 29.36 min.

FIG. 4A depicts HPLC profiles of extracts from recombinant Y. lipolytica with exogenous expression of α-carotene biosynthetic genes (carRP*, carB, FPPS::GGPPS, LCYe and LCYb) showing generation of putative α-carotene, β-carotene, γ-carotene, and δ-carotene. FIG. 4B depicts HPLC profiles of extracts from recombinant Y. lipolytica with exogenous expression of α-carotene biosynthetic genes (carRP*, carB, FPPS::GGPPS, LCYe and LCYb) showing generation of authentic α-carotene. FIG. 4C depicts HPLC profiles of extracts from recombinant Y. lipolytica with exogenous expression of carRP, carB, FPPS::GGPPS showing generation of putative β-carotene. FIG. 4D depicts HPLC profiles of extracts from recombinant Y. lipolytica with exogenous expression of carRP, carB, FPPS::GGPPS showing generation of authentic β-carotene.

FIG. 5A depicts UV spectre of samples extracted from recombinant lipolytica expressing α-carotene biosynthetic genes of putative α-carotene at 3.86 min. FIG. 5B depicts UV spectra of samples extracted from recombinant Y. lipolytica expressing of authentic α-carotene at 3.83 min. FIG. 5C depicts UV spectra of samples extracted from recombinant Y. lipolytica expressing putative β-carotene at 4.40 min. FIG. 50 depicts UV spectra of samples extracted from recombinant Y. lipolytica expressing of authentic β-carotene at 4.37 min, FIG. 5E depicts UV spectra of samples extracted from recombinant Y. lipolytica expressing putative γ-carotene at 5.20 min, FIG. 5F depicts UV spectra of samples extracted from recombinant Y. lipolytica expressing putative δ-carotene at 7.58 min.

FIG. 6A depicts HPLC profiles of extracts from Y. lipolytica with exogenous expression of lutein biosynthetic pathway genes (carRP*, carB, FPPS::GGPPS, LCYe, LCYb, BHY and CYP97C) showing generation of putative lutein. FIG. 6B depicts HPLC profiles of extracts from Y. lipolytica with exogenous expression of lutein biosynthetic pathway genes (carRP*, carB; FPPS::GGPPS, LCYe, LCYb, BHY and CYP97C) showing generation of authentic lutein. FIG. 6C depicts UV spectra of putative lutein peak at 12.46 min. FIG. 6D depicts UV spectra of authentic lutein peak at 12.40 min.

FIG. 7A depicts HPLC profiles of extracts from Y. lipolytica with exogenous expression of β-cryptoxanthin biosynthetic pathway genes (carRP, carB, FPPS::GGPPS and BCH1) showing generation of putative β-cryptoxanthin. FIG. 7B depicts UV spectra of putative β-cryptoxanthin peak at 9.83 min. FIG. 7C depicts putative β-carotene peak at 13.75 min.

FIG. 8A depicts positive ion APCI QTOF tandem mass spectrometry chromatogram of yeast extracts purified peak of α-carotene at 3.86 min. FIG. 8B depicts positive ion APCI QTOF tandem mass spectrometry chromatogram of yeast extracts purified peak of lutein at 12.28 min. FIG. 8C depicts positive ion APCI QTOF tandem mass spectrometry chromatogram of yeast extracts purified peak of β-cryptoxanthin at 9.83 min.

### DETAILED DESCRIPTION

The present disclosure is based in part on the discovery that industrially significant quantities of carotenoids and carotenoid products for commercial uses can desirably be produced in genetically modified microorganisms. More specifically, the inventors have discovered engineered pathways comprising specific combinations of biosynthetic enzymes from various organisms, wherein the combination of enzymes is capable of producing xanthophylls such as lutein, and β-cryptoxanthin. Advantageously, such pathways can be constructed in microorganisms to produce pure lutein, and β-cryptoxanthin without the low yield, and high labor costs of currently used methods. Additionally, the pathways can be constructed using nucleic acids encoding enzymes from microorganisms that do not carry any risk for humans and the environment, thereby providing a natural, safe alternative to chemical synthesis, and greater ease of isolation. As such, the present disclosure provides recombinant microorganisms encoding enzymes in pathways for producing pure xanthophylls such as lutein, and β-cryptoxanthin, and methods of using the recombinant microorganisms for producing such xanthophylls. The invention also provides methods of producing xanthphyll products, and methods of harvesting the xanthphyll products.

### I. Recombinant Microorganism

In one aspect, the present disclosure provides a recombinant microorganism capable of biosynthesizing one or more xanthophylls. A recombinant microorganism of the invention comprises at least one nucleic acid construct encoding one or more biosynthetic enzymes capable of producing xanthophylls. In particular, a recombinant microorganism of the present disclosure is capable of efficiently biosynthesizing industrially tractable quantities of xanthophylls, including δ-carotene, α-carotene, lutein, and β-cryptoxanthin. The microorganism, xanthophyll biosynthetic enzymes, and the genetic engineering of microorganisms to produce xanthophylls are discussed in more detail below.

### (a) Microorganisms

A recombinant microorganism of the present disclosure may be any microorganism provided the microorganism is generally regarded as safe for use in food or medical applications. In general, a microorganism of the disclosure is a bacterium, a fungus, or an alga. Preferably, a microorganism of the disclosure is a bacterium or a fungus. When selecting a particular microorganism for use in accordance with the present invention, it will generally be desirable to select a microorganism whose cultivation characteristics are amendable to commercial scale production. In general, any modifiable and cultivatable microorganism may be employed.

A microorganism may be naturally capable of producing xanthophylls. When a microorganism is naturally capable of producing xanthophylls, the microorganism may be genetically engineered to alter expression of one or more endogenous enzymes to enhance production of xanthophylls. In addition, when a microorganism is naturally capable of producing xanthophylls, the microorganism may be genetically engineered to express one or more exogenous enzymes to enhance production of xanthophylls. A microorganism may also be genetically engineered to alter expression of one or more endogenous genes, and to express one or more exogenous genes to enhance production of xanthophylls.

A suitable microorganism may be a fungal microorganism capable of producing xanthophylls. Fungal microorganisms that are naturally capable of producing xanthophylls are known in the art. Non-limiting examples of genera of fungi that are naturally capable of producing xanthophylls may include Blakeslea, Candida, Cryptococcus, Cunninghamella, Lipomyces, Marlierella, Mucor, Phycomyces, Pythium, Rhodosporidium, Rhodotorula, Trichosporon, and Yarrowia. Any fungus belonging to these genera may be utilized as host fungi according to the present invention, and may be engineered or otherwise manipulated to generate inventive, carotenoid and derivative producing fungal strains. Organisms of species that include, but are not limited to, Blakeslea trispora, Candida utilis, Candida pulcherrima, C. revkauji, C. tropicalis, Cryptococcus curvatus, Cunninghamella echinulata, C. elegans, C. japonica, Lipomyces starkeyi, L. lipoferus, Mortierella alpina, M. isabellina, M. ramanniana, M. vinacea, Mucor circinelloides, Phycomyces blakesleanus, Pythium irregulare, Rhodosporidium toruloides, Rhodotorula glutinis, R. gracilis, R.graminis, R. mucilaginosa, R. pinicola, Schizosaccharomyces pombe, Trichosporon pullans, T. cutaneum, Yarrowia lipolytica, and Xanthophyllomyces dendrorhous, may be used.

Alternatively, the fungus may not be naturally capable of producing xanthophylls. When the fungus is not naturally capable of producing xanthophylls, or produces limited amounts of xanthophylls, the fungus is genetically modified to express one or more exogenous genes to reconstruct or enhance a xanthophyll biosynthetic pathway for production of xanthophylls. Non-limiting examples of genera of fungi that are not naturally capable of producing xanthophylls, but that may be suitable for use in the present disclosure, may include Aspergillus, Botrytis, Cercospora, Fusarium (Gibberella), Kluyveromyces, Neurospora, Penicillium, Pichia (Hansenula), Puccinia, Saccharomyces, Schizosaccharomyces, Sclerotium, Trichoderma, and Xanthophyllomyces (Phaffia). Organisms of species that include, but are not limited to, Aspergillus nidulans, A. niger, A. terreus, Botrytis cinerea, Cercospora nicotianae, Fusarium fujikuroi (Gibberella zeae), Kluyveromyces lactis, K. lactis, Neurospora crassa, Pichia pastoris, Puccinia distincta, Saccharomyces cerevisiae, Sclerotium rolfsii, Schizosaccharomyces pombe, Trichoderma reesei, and Xanthophyllomyces dendrorhous (Phaffia rhodozyma), may be used.

A fungal microorganism of the disclosure may be Yarrowia lipolytica. Advantages of Y. lipolytica include, for example, tractable genetics and molecular biology, availability of genomic sequence (see, for example, Sherman et al., Nucleic Acids Res. 32 (Database issue):D315-8, 2004), suitability to various cost-effective growth conditions, and ability to grow to high cell density. Furthermore, there is already extensive commercial experience with Y. lipolytica.

Saccharomyces cerevisiae is also a useful host cell in accordance with the present invention, particularly due to its experimental tractability and the extensive experience that researchers have accumulated with the organism. Although cultivation of Saccharomyces under high carbon conditions may result in increased ethanol production, this can generally be managed by process and/or genetic alterations.

Other preferred fungal microorganisms of the disclosure may be Candida utilis, Pichia pastoris, Schizosaccharomyces pombe, Blakeslea trispora, and Xanthophyllomyces dendrorhous. The edible yeast C. utilis is an industrially important microorganism approved by the U.S. Food and Drug Administration as a safe substance. Through its large-scale production, C. utilis has become a promising source of single-cell protein as well as a host for the production of several chemicals, such as glutathione. P. pastoris is another non-carotenogenic yeast that has also been studied to production of carotenoids, and it is able to grow in organic materials.

A suitable microorganism may also be a bacterial microorganism capable of producing xanthophylls. Bacterial microorganisms that are naturally capable of producing xanthophylls are known in the art. Non-limiting examples of a bacterial microorganism capable of producing xanthophylls may include Erwinia species, and Agrobacterium aurantiacum.

Alternatively, the bacterium may not be naturally capable of producing xanthophylls. Non-limiting examples of genera of bacteria that are not naturally capable of producing xanthophylls, but that may be suitable for use in the present disclosure, may include Escherichia coli and Zymomonas mobilis. Escherichia coli and Zymomonas mobilis do not naturally synthesize xanthophylls, but by using carotenogenic genes, recombinant strains of such bacteria capable of accumulating carotenoids and their derivatives such as lycopene, beta-carotene, and astaxanthin have been produced.

A bacterial microorganism of the disclosure may be Escherichia coli, an intensively studied microorganism with tractable genetics that is also extensively used in industrial manufacturing for its suitability to various cost-effective growth conditions, and its ability to grow to high cell density.

Biosynthesis pathways of all xanthophylls of the invention comprise geranylgeranyl diphosphate (GGPP) as a starting point. As such, a preferred microorganism is a microorganism that is either naturally capable of producing GGPP, or is genetically modified to produce GGPP. A microorganism that is either naturally capable of producing GGPP, or is genetically modified to produce GGPP may be as described in International Patent Application No: PCT/US2016/023784, the disclosure of which is incorporated herein in its entirety. As described in International Patent Application No: PCT/US2016/023784, the choice of biosynthetic enzymes or combination of biosynthetic enzymes that are expressed in a microorganism can and will vary depending on the specific microorganism host cell or strain, and its ability to produce GGPP. An exemplary microorganism is Y. lipolytica genetically modified to produce GGPP as described in International Patent Application No: PCT/US2016/023784, the disclosure of which is incorporated herein in its entirety.

(b) Emzymes and Pathways

i. Biosynthetic Pathways of Xanthophylls

The carotenoid biosynthetic pathway begins with the formation of the C40-carbon phytoene from geranylgeranyl pyrophosphate (GGPP), followed by desaturation and isomerization reactions leading to synthesis of lycopene. Lycopene cyclases catalyze cyclization reactions of lycopene, which is a key branch point. Lycopene is cyclized to give rise to two branches, the β, ε branch and the β,β branch. The generation of α-carotene from the β, ε branch is dependent on lycopene ε-cyclase (LCYe) and lycopene β-cyclase (LCYb) and; the generation of β-carotene from the β,β branch is dependent on LCYb. Further hydroxylation of the carotenes leads to the biosynthesis of xanthophylls. Lutein is biosynthesized from α-carotene by the action of both β-ring and ε-ring hydroxylases, while β-cryptoxanthin is synthesized from β-carotene by only β-ring hydroxylase (BCH). Two different types of enzymes catalyzes these hydroxylation reactions, cytochromes P450 that belong to the CYP97 family, which catalyze the hydroxylations of α-carotene, and non-heme di-iron enzyme BHY as an ortholog of bacterial CrtZ, which catalyzes the hydroxylation of β-carotene.

According to the present invention, xanthophyll production in a host microorganism may be adjusted by modifying the expression or activity of one or more enzymes involved in xanthophyll biosynthesis. Such modification comprises expression of one or more heterologous nucleic acids encoding xanthophyll biosynthetic enzymes in the host cell. Alternatively or additionally, modifications may be made to the expression or activity of one or more endogenous or heterologous xanthophyll biosynthetic enzymes. A plurality of different heterologous xanthophyll biosynthetic enzymes may be expressed in the same host cell. This plurality may comprise only polypeptides from the same source organism (e.g., two or more sequences of, or sequences derived from, the same source organism). Alternatively, the plurality may include polypeptides independently selected from different source organisms (e.g., two or more sequences of, or sequences derived from, at least two independent source organisms).

Genetic modifications for producing, increasing production, or shifting production of xanthophylls described herein are described further below. A genetically modified microorganism may encode any of the xanthophyll biosynthetic enzymes, but with some further modifications designed to enhance production of the xanthophylls.

As described above, the selection of the organism of origin of the enzyme may be important and is preferably an organism generally regarded as safe. Non-limiting examples of organisms of origin of metabolic enzymes that may be regarded as safe include Mucor circinelloides, Phycomyces blakesleeanus, Y. lipolytica, Saccharomyces cerevisiae, Candida utilis, Pichia pastoris, and Schizosaccharomyces pombe. Preferably, the microorganism is Y. lipolytica.

ii. δ-Carotene, α-Carotene, and Lutein

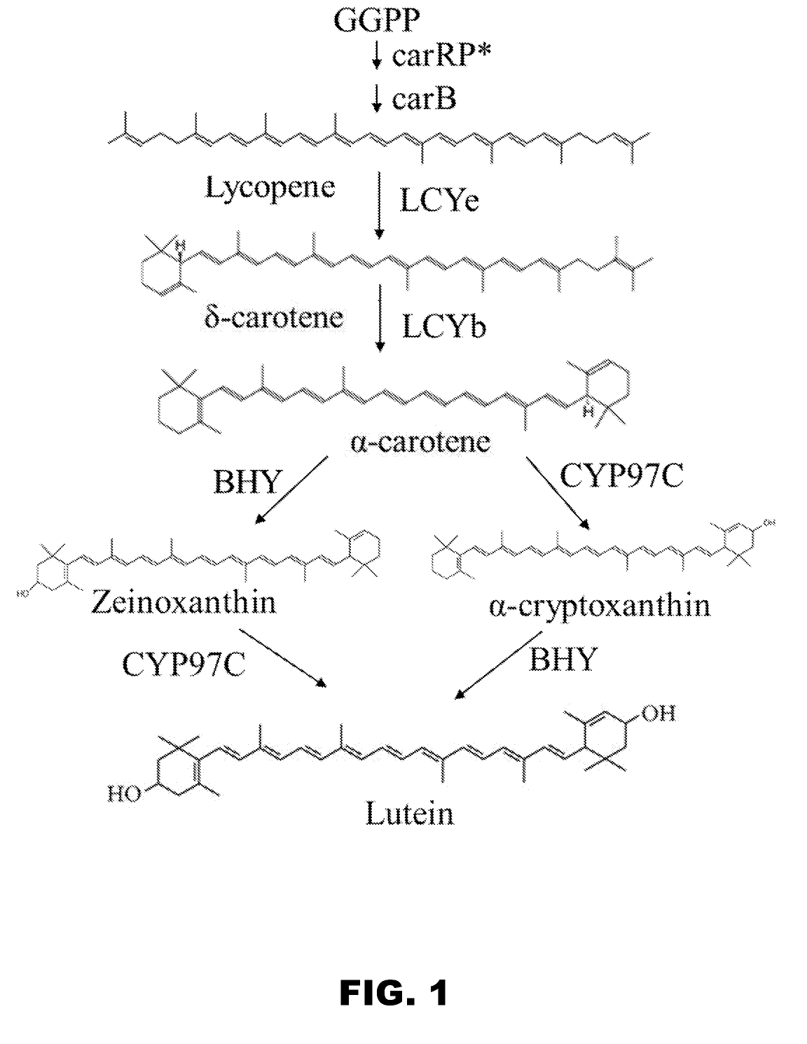

In some aspects, a microorganism of the present disclosure is a recombinant microorganism genetically engineered to produce or increase production of δ-carotene, α-carotene, or lutein. Preferably, δ-carotene, α-carotene, or lutein are produced using the pathway shown in FIG. 1. As shown in FIG. 1, production of δ-carotene, α-carotene, or lutein from GGPP starts with phytoene synthase (PSase), and phytoene dehydrogenase to produce lycopene, from which all xanthophylls of the invention are produced. Lycopene ε-cyclase (LCYe) produces δ-carotene from lycopene. Lycopene β-cyclase (LCYb) produces α-carotene from δ-carotene. β-carotene hydroxylase (CYP97A or BHY) and P450 carotene ε-ring hydroxylase (CYP97C) produce lutein from α-carotene. These enzymes are referred to herein as xanthophyll biosynthetic enzymes.

As such, a recombinant microorganism of the present disclosure may be genetically engineered to express PSase and phytoene dehydrogenase to produce lycopene from GGPP, and further express any combination of one or more of the xanthophyll biosynthetic enzymes of the pathway shown in FIG. 1. For instance, a microorganism may be genetically engineered to express PSase and phytoene dehydrogenase to produce lycopene from GGPP, and further express LCYe for production of β-carotene from GGPP; further express LCYe, and LCYb for production of α-carotene; or further express LCYe, LCYb, BHY, and CYP97C for production of lutein.

Preferably, a microorganism of the invention is genetically engineered to express PSase and phytoene dehydrogenase to produce lycopene from GGPP, and further express any combination of one or more of the xanthophyll biosynthetic enzymes of the pathway shown in FIG. 1. A preferred PSase enzyme comprises the phytoene synthase activity encoded by the P domain of the carRP gene of M. circinelloides. More preferably, when the carRP gene of M. circinelloides is used as a source of the PSase enzyme activity for producing lycopene, the carRP gene is modified to decrease or inhibit lycopene cyclase activity encoded by the R domain of the carRP gene (carRP*). As used herein, the term “decrease or inhibit” refer to a substantial or complete elimination of the activity of an enzyme such as lycopene cyclase. As such, decreasing or inhibiting the lycopene cyclase activity of the carRP gene of M. circinelloides prevents or substantially reduces the cyclization of the lycopene to γ-carotene, and ensures the accumulation of lycopene in the microorganism. More preferred, the codon-optimized modified carRP gene of M. circinelloides (carRP*) encoded by SEQ ID NO: 30 is used as a source of the PSase enzyme activity for producing lycopene.

A preferred phytoene dehydrogenase enzyme comprises phytoene dehydrogenase encoded by the carB gene of M. circinelloides. Preferably, the codon-optimized carB gene of M. circinelloides encoded by SEQ ID NO: 26 is used as a source of the phytoene dehydrogenase enzyme for producing lycopene.

In some embodiments, a microorganism is genetically engineered to express LCYe for production of δ-carotene from lycopene. Preferably, the LCYe enzyme comprises a Marchantia polymorpha LCYe. More preferably, the LCYe enzyme comprises a Marchantia polymorpha LCYe having SEQ ID NO.: 35.

In other embodiments, a microorganism is genetically engineered to express LCYe and further express LCYb for production of α-carotene from lycopene. LCYe may be as described above. Preferably, the LCYb enzyme comprises an LCYb enzyme selected from LCYb from Chlamydomonas reinhardtii, an LCYb enzyme from Chromochloris zofingiensis, and a combination thereof. More preferably, a microorganism is genetically engineered to further express an LCYb selected from LCYb from Chlamydomonas reinhardtii having SEQ ID NO.: 37 and a LCYb from Chromochloris zofingiensis having SEQ ID NO.: 41, for production of α-carotene from lycopene.

In yet other embodiments, a microorganism is genetically engineered to express LCYe, LCYb, and further express BHY, and CYP97C for production of lutein from lycopene. LCYe and LCYb may be as described above. Preferably, the BHY enzyme and the CYP97C enzyme are from Marchantia polymorpha. More preferably, a microorganism is genetically engineered to further express a Marchantia polymorpha BHY having SEQ ID NO.: 44 and a Marchantia polymorpha CYP97C having SEQ ID NO.: 47, for production of lutein from lycopene.

It will be recognized that the genetic modifications described herein for producing the various xanthophylls may be in addition to any or all of the genetic modifications described above for producing GGPP and/or lycopene. Preferably, when the genetically engineered microorganism is Y. lipolytica, the genetic modifications for producing GGPP and/or lycopene may be as described in International Patent Application No: PCT/US2016/023784.

### iii. β-Cryptoxanthin

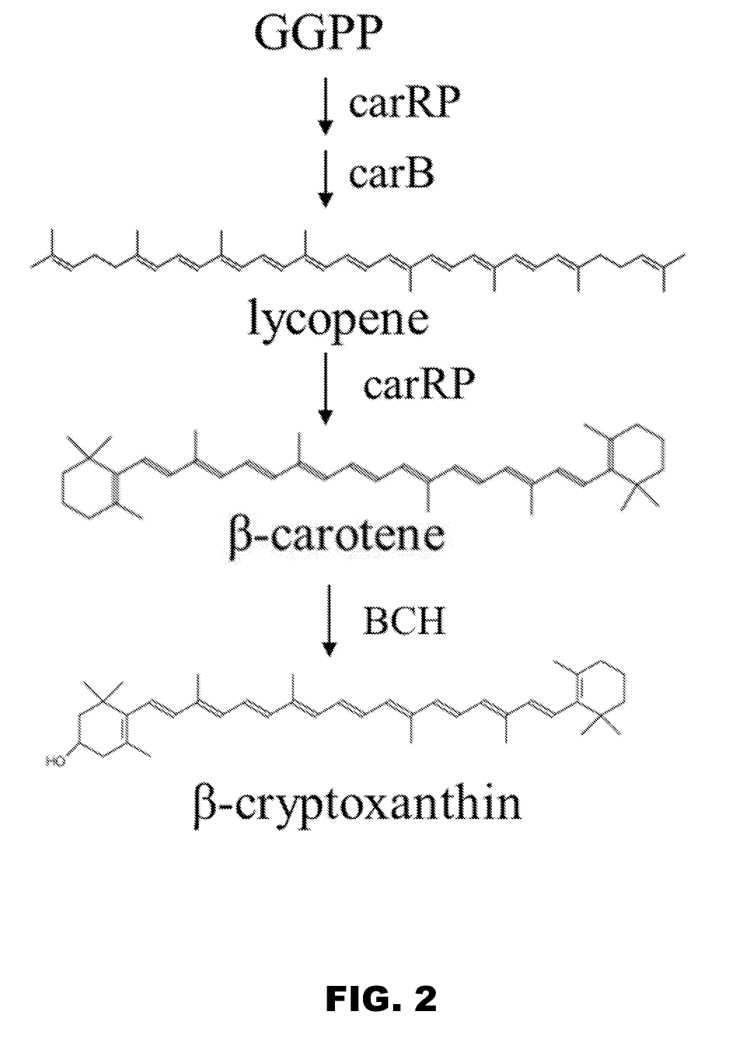

In other aspects, a microorganism of the present disclosure may be genetically engineered to produce or increase production of β-cryptoxanthin. Production of β-cryptoxanthin from lycopene may be produced using the pathway shown in FIG. 2. As shown in FIG. 2, production of β-cryptoxanthin from GGPP starts with PSase and phytoene dehydrogenase to produce lycopene. Lycopene cyclase and β-carotene hydroxylase (BCH) then produce β-cryptoxanthin. As such, a microorganism of the present disclosure may be genetically engineered to express PSase and phytoene dehydrogenase to produce lycopene from GGPP, and further express lycopene cyclase and BCH to produce β-cryptoxanthin. Alternatively, if the microorganism is naturally capable of producing sufficient amounts of lycopene, a recombinant microorganism of the present disclosure may be genetically engineered to express lycopene cyclase and BCH but not the biosynthetic enzymes for producing lycopene to produce β-cryptoxanthin. Preferably, the PSase enzyme comprises the phytoene synthase activity encoded by the R domain of the carRP gene of M. circinelloides. More preferred, the codon-optimized carRP gene of M. circinelloides is used as a source of the phytoene dehydrogenase enzyme for producing lycopene.

Preferably, the phytoene dehydrogenase enzyme comprises the phytoene dehydrogenase encoded by the carB gene of M. circinelloides. More preferred, the codon-optimized carB gene of M. circinelloides encoded by SEQ ID NO: 26 is used as a source of the phytoene dehydrogenase enzyme for producing lycopene.

The lycopene cyclase enzyme preferably comprises the lycopene cyclase encoded by the P domain of the carRP gene of M. circinelloides. More preferably, the codon optimized P domain of the carRP gene of M. circinelloides is used as a source of lycopene cyclase enzyme.

When the microorganism is genetically engineered to produce or increase production of β-cryptoxanthin, the BCH enzyme preferably comprises the BCH enzyme encoded by the GmBCH gene of Glycine max. More preferably, the BCH enzyme preferably comprises the BCH enzyme encoded by the codon optimized GmBCH gene of Glycine max having SEQ ID NO.: 50.

It will be recognized that the genetic modifications described herein for producing the various xanthophylls may be in addition to any or all of the genetic modifications described above for producing lycopene.

### (c) Genetic Engineering

According to the present invention, xanthophyll production in a host organism may be adjusted by expressing or modifying the expression or activity of one or more proteins involved in xanthophyll biosynthesis. Such modification may involve introduction of at least one nucleic acid construct comprising one or more nucleic acid sequences encoding heterologous xanthophyll biosynthesis polypeptides into the host microorganism. Alternatively or additionally, modifications may be made to the expression or activity of one or more endogenous or heterologous xanthophyll biosynthesis polypeptides. Given the considerable conservation of components of the xanthophyll biosynthesis polypeptides, it is expected that heterologous xanthophyll biosynthesis polypeptides will often function even in significantly divergent organisms. Furthermore, should it be desirable to introduce more than one heterologous xanthophyll biosynthesis polypeptide, in many cases polypeptides from different source organisms will function together.

At least one nucleic acid construct encoding a plurality of different heterologous xanthophyll biosynthesis polypeptides may be introduced into the same host cell. A plurality of different heterologous xanthophyll biosynthesis polypeptides may comprise only polypeptides from the same source organism (e.g., two or more sequences of, or sequences derived from the same source organism). Alternatively, a plurality of different heterologous xanthophyll biosynthesis polypeptides may comprise polypeptides independently selected from different source organisms (e.g., two or more sequences of, or sequences derived from, at least two independent source organisms).

Those of ordinary skill in the art will appreciate that the selection of a particular microorganism for use in accordance with the present invention will also affect, for example, the selection of expression sequences utilized with any heterologous polypeptide to be introduced into the cell, and will also influence various aspects of culture conditions, etc. Much is known about the different gene regulatory requirements, protein targeting sequence requirements, and cultivation requirements of different host cells to be utilized in accordance with the present invention (see, for example, with respect to Yarrowia, Barth et al. FEMS, Microbiol Rev. 19:219, 1997; Madzak et al., J. Biotechnol. 109:63, 2004; see, for example, with respect to Xanthophyllomyces, Verdoes et al., Appl Environ Microbiol. 69: 3728-38, 2003; Visser et al. FEMS Yeast Res 4: 221-31, 2003; Martinez et al., Antonie Van Leeuwenhoek. 73(2):147-53, 1998; Kim et al. Appl Environ Microbiol. 64(5):1947-9, 1998; Wery et al., Gene 184(1):89-97, 1997; see, for example, with respect to Saccharomyces, Guthrie and Fink, Methods in Enzymology 194:1-933, 1991). In certain aspects, for example, targeting sequences of the host cell (or closely related analogs) may be useful to include for directing heterologous proteins to subcellular localization. Thus, such useful targeting sequences can be added to heterologous sequences for proper intracellular localization of activity. In other aspects (e.g., addition of mitochondrial targeting sequences), heterologous targeting sequences may be eliminated or altered in the selected heterologous sequences (e.g., alteration or removal of source organism plant chloroplast targeting sequences).

As described above, a recombinant microorganism of the present disclosure comprises at least one nucleic acid construct comprising one or more nucleic acid sequences encoding a xanthophyll biosynthesis enzyme. A nucleic acid sequence of the present disclosure may be operably linked to one or more expression control sequences for expressing a xanthophyll biosynthesis enzyme. “Expression control sequences” are regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, that have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A recombinant microorganism may synthesize one, two, three, four, five, or more xanthophyll biosynthetic enzymes. A one or more nucleic acid encoding any of the enzymes disclosed herein may be chromosomally integrated, or may be expressed on an extrachromosomal vector. Suitable vectors are known in the art. Similarly, methods of chromosomally inserting a nucleic acid are known in the art. For additional details, see the Examples.

A large number of promoters, including constitutive, promoters for high-level expression (overexpression), inducible and repressible promoters, from a variety of different sources are well known in the art. Representative sources include, for example, viral, mammalian, insect, plant, yeast, and bacterial cell types, and suitable promoters from these sources are readily available, or can be made synthetically based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction).

Non-limiting examples of suitable promoters may include an intron-containing transcriptional elongation factor TEF promoter (TEFIN), GPAT (glycerol-3-phosphate o-acyl transferase), YAT1 (ammonium transporter), EXP1 (export protein), and GPD (glyceraldehyde-3-phosphate dehydrogenase), FBA1(fructose 1,6-bisphosphate aldolase), GPM1 (phosphoglycerate mutase), FBA1 IN (FBA1 containing an intron), the GAL promoters of yeast, and hp4d (Four tandem copies of upstream activator sequences (UAS1B) fragment from pXPR2 and a minimal pLEU2 fragment. Preferably, a promoter suitable for overexpression of proteins is used to overexpress one or more xanthophyll biosynthesis enzymes of the disclosure. Non-limiting examples of suitable promoters for overexpression of proteins include intron-containing transcriptional elongation factor TEF promoter (TEFIN) and EXP1 (export protein).

The nucleic acid sequences are operably linked to one or more expression control sequences. One or more of the nucleic acid sequences may be operably linked to an intron-containing transcriptional elongation factor TEF promoter (TEFIN). Alternatively, one or more of the nucleic acid sequences may be operably linked to an export protein promoter (EXP1). The nucleic acid construct may be codon-optimized for expression in a heterologous microorganism.

A nucleic acid construct of the invention may comprise a plasmid suitable for use in a microorganism of choice. Such a plasmid may contain multiple cloning sites for ease in manipulating nucleic acid sequences. Numerous suitable plasmids are known in the art.

### II. Methods

In another aspect, the present disclosure provides a method of producing xanthophylls. Preferably, a method of the present disclosure is capable of producing lycopene, carotene, and ionones. Most preferred are methods of producing α-ionone and β-ionone.

A method of the disclosure comprises cultivating a recombinant microorganism expressing xanthophyll biosynthesis enzymes under conditions sufficient for the production of the xanthophyll. A recombinant microorganism may be as described in Section I above.

As discussed above, production of xanthophylls in a recombinant microorganism of the present disclosure generally comprises cultivating the relevant organism under conditions sufficient to accumulate a xanthophyll, harvesting the modified microorganism, and isolating the xanthophyll from the harvested microorganism.

Methods of cultivating a microorganism are well known in the art and may be similar to conventional fermentation methods. As will be appreciated by a skilled artisan, the culture conditions sufficient to accumulate a xanthophyll can and will vary depending on the specific microorganism host cell or strain and the xanthophyll produced by the microorganism. A recombinant microorganism may be cultured in a medium comprising a carbon source, a nitrogen source, and minerals, and if necessary, appropriate amounts of nutrients which the microorganism requires for growth. As the carbon source, saccharides such as glucose, fructose, sucrose, molasses and starch hydrolysate, organic acids such as fumaric acid, citric acid and succinic acid, or alcohol such as ethanol and glycerol may be used. As the nitrogen source, various ammonium salts such as ammonia and ammonium sulfate, other nitrogen compounds such as amines, a natural nitrogen source such as peptone, soybean-hydrolysate, or digested fermentative microorganism may be used. As minerals, potassium monophosphate, magnesium sulfate, sodium chloride, ferrous sulfate, manganese sulfate, calcium chloride, and the like may be used. As vitamins, thiamine, yeast extract, and the like, may be used. The pH of the medium may be between about 5 and about 9. When the microorganism comprises a mutation that limits the production of an essential nutrient, the medium may be supplemented with the essential nutrient to maintain growth of the microorganism.

When the microorganism is Y. lipolytica or S. cerevisiae, the recombinant microorganism may be cultivated in YPD medium (10 g/L yeast extract, 20g/L peptone and 20 g/L glucose) to produce a xanthophyll of the disclosure. Y. lipolytica or S. cerevisiae may also be cultivated in SD-dropout medium containing 1.7 g/L yeast nitrogen base without amino acids and ammonium sulphate, 20 g/L D-glucose, 5 g/L ammonium sulphate, 2 g/L yeast synthetic drop-out medium supplements and other nutrients that may vary depending on the nutrient requirement of the Y. lipolytica or S. cerevisiae strain.

Various temperature and duration of cultivation may also be used and will vary depending on the specific microorganism host cell or strain, the xanthophyll produced by the microorganism, and its culture conditions. The cultivation may be performed under aerobic conditions, such as by shaking and/or stirring with aeration. When the microorganism is Y. lipolytica or S. cerevisiae, a recombinant microorganism may be cultivated at a temperature of about 20 to about 40° C., preferably at a temperature of about 20, 21, 22, 23, 24, 25, 26, 27, 28, 29, 30, 31, 32, 33, 34, 35, 36, 37, 38, 39, and about 40° C. More preferably, a recombinant Y. lipolytica or S. cerevisiae may be cultivated at a temperature of about 28° C.

A recombinant microorganism of the present disclosure may be cultivated for about 1, 2, 3, 4, 5, 6, 7, 8, 9, 10 or more days before isolating xanthophyll. Preferably, when a recombinant microorganism is Y. lipolytica, the recombinant microorganism is cultivated for about 1, 2, or 3 days before isolating xanthophylls, preferably, 1 day.

When a recombinant microorganism is E. coli, the microorganism may be cultivated in LB medium in a shaker at a temperature of about 25 to about 40° C., preferably at a temperature of about 37° C. If carotenogenic enzymes expressed in E. coli are under the control of an inducible promoter, the enzymes may be induced at a temperature of about 25 to 35° C., preferably at a temperature of about 30° C.

Methods and systems for isolating xanthophylls have been established for a wide variety of xanthophylls (see, for example, Perrut M, Ind Eng Chem Res, 39: 4531-4535, 2000, the disclosure of which is incorporated herein in its entirety). In brief, cells are typically recovered from culture, often by spray drying, filtering or centrifugation. In some instances, cells are homogenized and then subjected to supercritical liquid extraction or solvent extraction (e.g., with solvents such as chloroform, hexane, methylene chloride, methanol, isopropanol, ethyl acetate, etc.) using conventional techniques.

Given the sensitivity of xanthophylls generally to oxidation, the disclosure may employ oxidative stabilizers (e.g., tocopherols, vitamin C; ethoxyquin; vitamin E, BHT, BHA, TBHQ, etc, or combinations thereof) during and/or after xanthophyll isolation. Alternatively or additionally, microencapsulation, for example with proteins, may be employed to add a physical barrier to oxidation and/or to improve handling (see, for example, U.S. Patent Application 2004/0191365).

In general, a recombinant microorganism accumulate xanthophylls to levels that are greater than at least about 0.1% of the dry weight of the cells. The total xanthophyll accumulation in a recombinant microorganism may be to a level at least about 1° A, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20% or more of the total dry weight of the cells.

### Definitions

When introducing elements of the present disclosure, the articles “a,” “an,” “the,” and “said” are intended to mean that there are one or more of the elements. The use of or means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Unless otherwise defined herein, scientific and technical terms used in connection with the present disclosure shall have the meanings that are commonly understood by those of ordinary skill in the art. The meaning and scope of the terms should be dear, however, in the event of any latent ambiguity, definitions provided herein take precedent over any dictionary or extrinsic definition. Further, unless otherwise required by context, singular terms as used herein and in the claims shall include pluralities and plural terms shall include the singular.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and encompass a variety of yeast or fungal strains that may be utilized as host strains to produce carotenoids and their derivatives. Thus, the terms “transformants” and “transfectants” include the primary subject cell and cell lines derived therefrom without regard for the number of transfers.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis,

The term “expression cassette” refers to a nucleic acid comprising the coding sequence of a selected gene and regulatory sequences preceding (expression control sequences) and following (non-coding sequences) the coding sequence that are required for expression of the selected gene product. Thus, an expression cassette is typically composed of: (1) a promoter sequence; (2) a coding sequence (i.e., ORF); and (3) a 3′ untranslated region (i.e., a terminator) that, in eukaryotes, usually contains a polyadenylation site. The expression cassette(s) is usually included within a vector to facilitate cloning and transformation. Different expression cassettes can be transformed into different organisms including bacteria, yeast, plants and mammalian cells, as long as the correct regulatory sequences are used for each host.

“Expression control sequences” are regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, that have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise expression control sequences (i.e., non-coding) as well as coding sequences and introns. The transcribed region of the gene may also include untranslated regions including introns, a 5′-untranslated region (5′-UTR) and a 3′-untranslated region (3′-UTR).

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The terms “operably linked”, “operatively linked,” or “operatively coupled” as used interchangeably herein, refer to the positioning of two or more nucleotide sequences or sequence elements in a manner which permits them to function in their intended manner. A nucleic acid molecule according to the invention may include one or more DNA elements capable of opening chromatin and/or maintaining chromatin in an open state operably linked to a nucleotide sequence encoding a recombinant protein. A nucleic add molecule may additionally include one or more DNA or RNA nucleotide sequences chosen from: (a) a nucleotide sequence capable of increasing translation, (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein, it is not necessarily contiguous with it. Operable linking of various nucleotide sequences is accomplished by recombinant methods well known in the art, e.g., using PCR methodology, by ligation at suitable restriction sites, or by annealing. Synthetic oligonucleotide linkers or adaptors can be used in accord with conventional practice if suitable restriction sites are not present.

The terms “polynucleotide,” “nucleotide sequence” and “nucleic acid” are used interchangeably herein, and refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase, Prokaryotic promoters contain Shine-Dalgarno sequences in addition to the -10 and -35 consensus sequences.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

### EXAMPLES

The publications discussed above are provided solely for theft disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1

### Construction of Expression Vectors for Lycopene Production in Yarrowia lipolytica

Two genes are required for lycopene production, namely phytoene desaturase and phytoene synthase which convert geranylgeranyl diphosphate (GGPP) to lycopene in Yarrowia lipolytica. The genes were selected from M. circinelloides, Phycomyces blakesleeanus or Xanthophyllomyces dendrorhous (International Patent Application No: PCT/US2016/023784) and codon-optimized for expression in Yarrowia lipolytica.

Yarrowia lipolytica expression vector was constructed as follows: Plasmid YAL-zeta-URA3-TEF-XPR2 was constructed based on integration vector YAL-rDNA-URA3-TEF-XPR2 (US patent application No: PCT/US2016/023784). A 315 bp nucleic acid fragment comprising the recombination site zetal and a 391 bp nucleic acid fragment comprising the recombination site zeta2 were amplified using primers zetal-NdeI-NotI-F (SEQ ID NO: 1) and Zeta1-SphIR (SEQ ID NO: 2), and Zeta2-SalI-ACSIF (SEQ ID NO: 3) and Zeta2-AflIII-NotIR (SEQ ID NO: 4), respectively, using Y. lipolytica genomic DNA as a template. The plasmid YAL-rDNA-URA3-TEF-XPR2 was cut by NdeI and SphI, the fragment zetal was then cloned into the NdeI/SphI restriction sites of the YAL-rDNA-URA3-TEF-XPR2 construct to yield YAL-zeta1-URA3-TEF-XPR2. The plasmid YAL-zeta1-URA3-TEF-XPR2 was cut by SalI and AflIII and the fragment zeta2 was cloned into the SalI and AflIII restriction sites of YAL-zeta1-URA3-TEF-XPR2 to form the final plasmid YAL-zeta-URA3-TEF-XPR2.

The pathway of lycopene biosynthesis was reconstituted in Y. lipolytica by over-expressing three enzymes: phytoene dehydrogenase (carB), mutated bifunctional lycopene cyclase/phytoene synthase (carRP*:K78E and P216S in wild type carRP) and the fusion gene of FPPS::GGPPS. The three genes, codon-optimized OptcarB (SEQ ID NO: 26), codon-optimized OptcaRP* (SEQ ID NO: 30), and FPPS::GGPPS (SEQ ID NO: 32), flanked with BamHI and AvrII, were amplified by PCR using the primers, OptcarB-BamHIF (SEQ ID NO: 5) and OptcarB-AvrIIR (SEQ ID NO: 6), OptcarRP*-BamHIF (SEQ ID NO: 7) and OptcarRP*-AvrIIR (SEQ ID NO: 8), and FPPS::GGPPS-BamHIF (SEQ ID NO: 9) and FPPS::GGPPS-AvrIIR (SEQ ID NO: 10), respectively. The three nucleotide fragments were then digested with BamHI/AvrII and ligated to the BamHI/AvrII-digested YAL-zeta-URA3-TEF-XPR2 vector to form the plasmids YAL-zeta-URA3-TEF-OptcarB, YAL-zeta-URA3-TEF-OptcarRP*, and YAL-zeta-URA3-TEF-FPPS::GGPPS, respectively. TEF-OptcarRP*-XPR2 and TEF-FPPS::GGPPS-XPR2 cassettes were obtained by PCR amplification with primers PromTEF-SalIF (SEQ ID NO: 11) and TermXPR2-ASCIR (SEQ ID NO: 12) and PromTEF-ASCIF (SEQ ID NO: 13) and TermXPR2-ASCIR (SEQ ID NO: 12), respectively. First, the TEF-OptcarRP*-XPR2 was cloned into the SalI/AscI restriction sites of the YAL-zeta-URA3-TEF-OptcarB vector to generate the YAL-zeta-URA3-TEF-OptcarB-TEF-OptcarRP*plasmid. Second, YAL-zeta-URA3-TEF-OptcarB-TEF-OptcarRP* was digested using AscI and treated with Antarctic Phosphatase following the manufacturer's manual (New England Biolabs, Ipswich, Mass.). The amplified AscI-digested TEF-FPPS::GGPPS-XPR2 cassette was then cloned into the AscI-digested YAL-zeta-URA3-TEF-OptcarB-TEF-OptcarRP* to generate YAL-zeta-URA3-TEF-OptcarB-TEF-OptcarRP*-TEF-FPPS::GGPPS.

### Example 2

### Construction of Yarrowia lipolytica Strains for the Production of Lycopene

Plasmid YAL-zeta-URA3-TEF-OptcarB-TEF-OptcarRP*-TEF-FPPS::GGPPS was digested with NotI and the 10 kb fragment was gel purified. The fragment was used to transform Yarrowia lipolytica CLIB138 host and select on minimal media plate without uracil. The pink colonies were grown in 5 ml YPD medium for 4 days at 30° C. and extracted for further HPLC analysis. The colony with highest lycopene content named as LY-1 and was chosen for further analysis.

### Example 3

### Extraction of Lycopene and HPLC Method Development

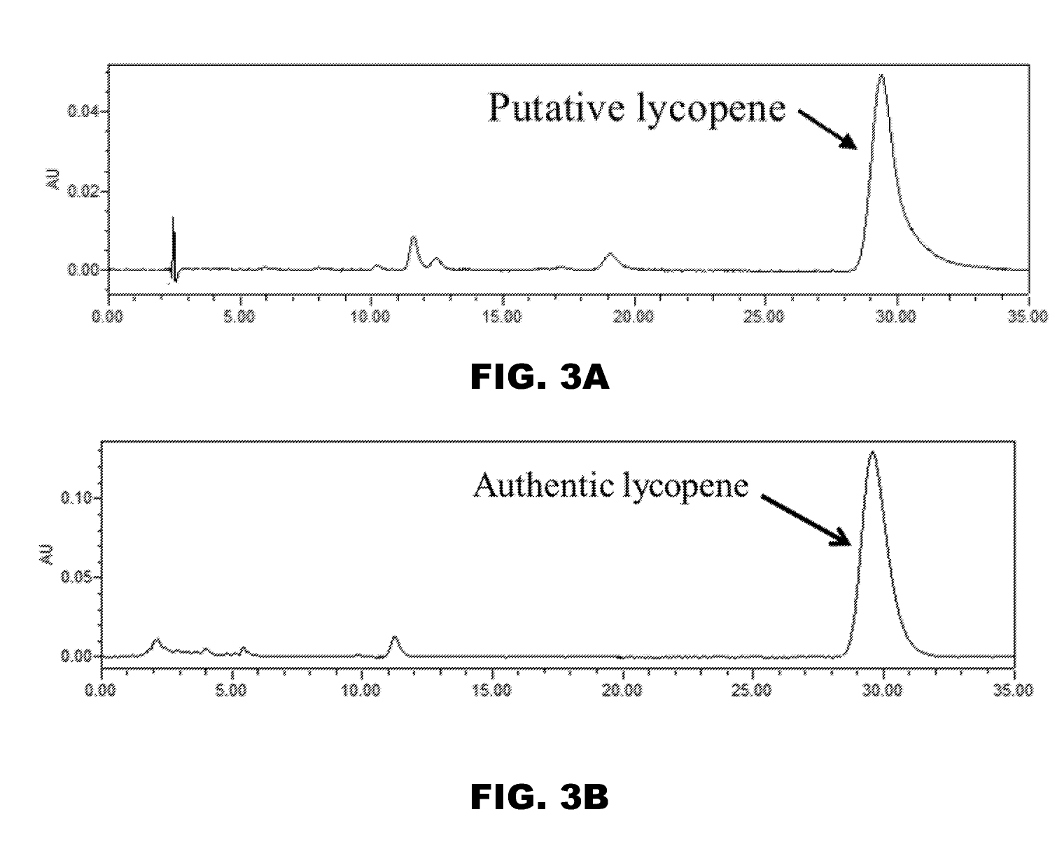

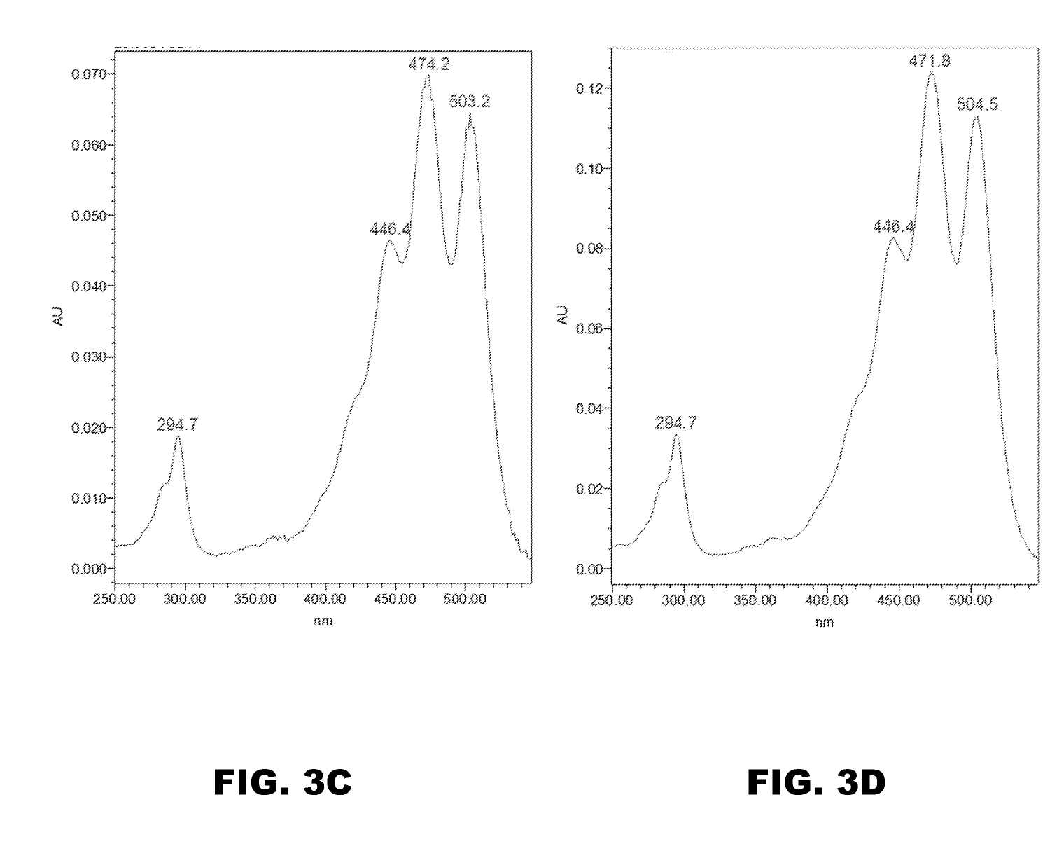

After four days of growth, 1 ml of cell culture was harvested by centrifugation and the cell pellet was suspended in 1 ml 100% ethanol for 30 min at 50° C., then centrifugation and the cells pellet was extracted with 1 ml ethyl acetate. The mixture was incubated at 50° C. in hot water bath for 30 min and vortexed every 5 min. Then the mixture was centrifuged for 10 min at 15,000 rpm and the supernatant was transferred into a new tube. The process was repeated three times and the supernatants were pooled and concentrated till 50% of the volume and then chilled at 4° C. in cold water bath for two hour for the crystallization of lycopene. The mixture is centrifuged to recover crystal for HPLC analysis. The HPLC analysis of lycopene was carried out using an Alliance 2996 HPLC (Waters) equipped with a 2476 photodiode array detector. Samples were separated by reverse-phase chromatography on a YMC carotenoid column (particle size 5 μm; 250×4.6 mm) isocratically using a mobile phase of methyl-t-butyl ether: methanol: ethyl acetate (40:50:10, v/v/v) at a flow rate of 1.5 ml/min. Peaks were measured at a wavelength from 250-600 nm to facilitate the detection of lycopene. As shown in FIGS. 3A, 3B, 3C, and 3D, the Y. lipolytica carrying carRP*, carB and FPPS::GGPPS genes accumulates lycopene by comparing the retention time and UV spectrum with authentic lycopene standard.

### Example 4

### Construction of α-Carotene Biosynthetic Pathway in Yarrowia lipolytica

The conversion of lycopene to α-carotene involves two enzymes, lycopene ε-cyclase (LCYe) and lycopene β-cyclase (LCYb) (FIG. 1). But this conversion typically leads to the synthesis of β-carotene, so it's necessary to identify a combination of LCYe and LCYb enzymes which can convert lycopene to α-carotene efficiently without or with minimal accumulation of β-carotene. The different LCYe and LCYb genes from algae and plants were screened in Yarrowia lipolytica.

The coding sequences of LCYe gene from Marchantia polymorpha in combination with the coding sequences of LCYb gene from Chlamydomonas reinhardtii, or Chromochloris zofingiensis showed maximal accumulation of α-carotene. The first 47 amino acid residues of lycopene ε-cyclase from Marchantia polymorpha (tMpLCYe; SEQ ID NO: 34) were the signal peptide to the chloroplast. The signal peptide sequences of MpLCYe was removed and the remaining coding sequence was synthesized based on Yarrowia lipolytic preferred codon usage (SEQ ID NO: 35) and amplified using primers tMpLCYe-BamHIF (SEQ ID NO: 14) and tMpLCYe-AvrIIR (SEQ ID NO: 15), and cloned into BamHI and AvrII restriction sites of YAL-zeta-URA3-TEF-XPR2 vector, to generate YAL-zeta-URA3-TEF-MpLCYE.

Similarly, lycopene β-cyclase (CrLCYb; SEQ ID NO: 37) from Chlamydomonas reinhardtii was synthesized and amplified by tCrLCYb-BamHIF (SEQ ID NO: 38) and tCrLCYb-AvrIIR (SEQ ID NO: 16) and CzLCYb (SEQ ID NO: 40) from Chromochloris zofingiensis was synthesized (SEQ ID NO: 41) and amplified by tCzLCYb-BamHIF (SEQ ID NO: 18) and tCzLCYb-AvrIIR (SEQ ID NO: 19) and cloned into BamHI and AvrII restriction sites of YAL-zeta-URA3-TEF-XPR2 to give rise to YAL-zeta-URA3-TEF-CrLCYb and YAL-zeta-URA3-TEF-CzLCYb.

TEF-CrLCYb-XPR2 and TEF-CzLCYb-XPR2 cassettes were obtained by PCR amplification with primers PromTEF-SalIF (SEQ ID NO: 11) and TermXPR2-ASCIR (SEQ ID NO: 12). The TEF-CrLCYb-XPR2 or TEF-CzLCYb-XPR2 was cloned into the SalI/AscI restriction sites of the YAL-zeta-URA3-TEF-MpLCYe vector to generate the YAL-zeta-URA3-TEF-MpLCYe-TEF-CrLCYb and YAL-zeta-URA3-TEF-MpLCYe-TEF-CzLCYb plasmids.

### Example 5

### Construction of Yarrowia lipolytica Strains for the Production of α-Carotene

Lycopene-producing strain LY-1 was transformed with plasmid YAL-LEU2-Cre to excise URA3 selection marker according to the method described in US patent application Publication No PCT/US2016/023784. The resulting lycopene-producing strain without URA3 marker gene was designated LY-2. Plasmid YAL-zeta-URA3-TEF-MpLCYe-TEF-CrLCYb and YAL-zeta-URA3-TEF-MpLCYe-TEF-CzLCYb were cut by NotI to extract large fragment containing LCYe-LCYb cassette. The cassette was introduced into LY-2 strain host and plated on minimal media plate without uracil supplementation. Those red-orange colonies was inoculated into 5 ml YPD medium and extracted for HPLC analysis.

### Example 6

### Production of α-Carotene in Yarrowia lipolytica

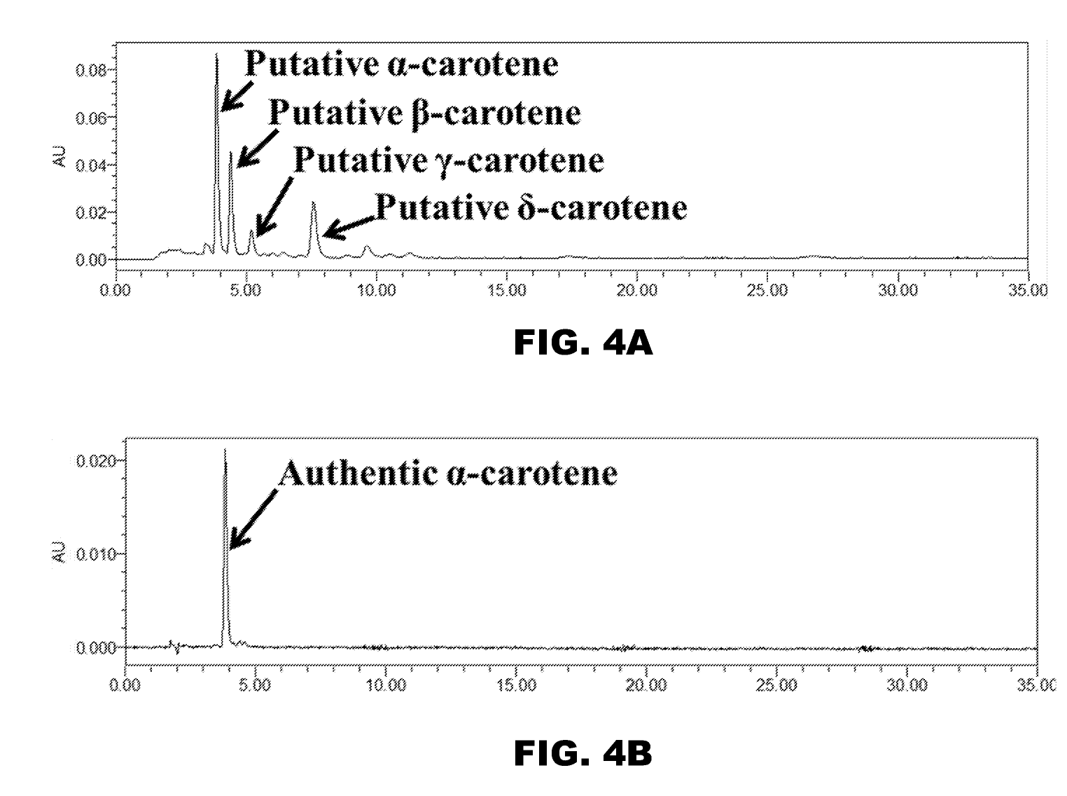

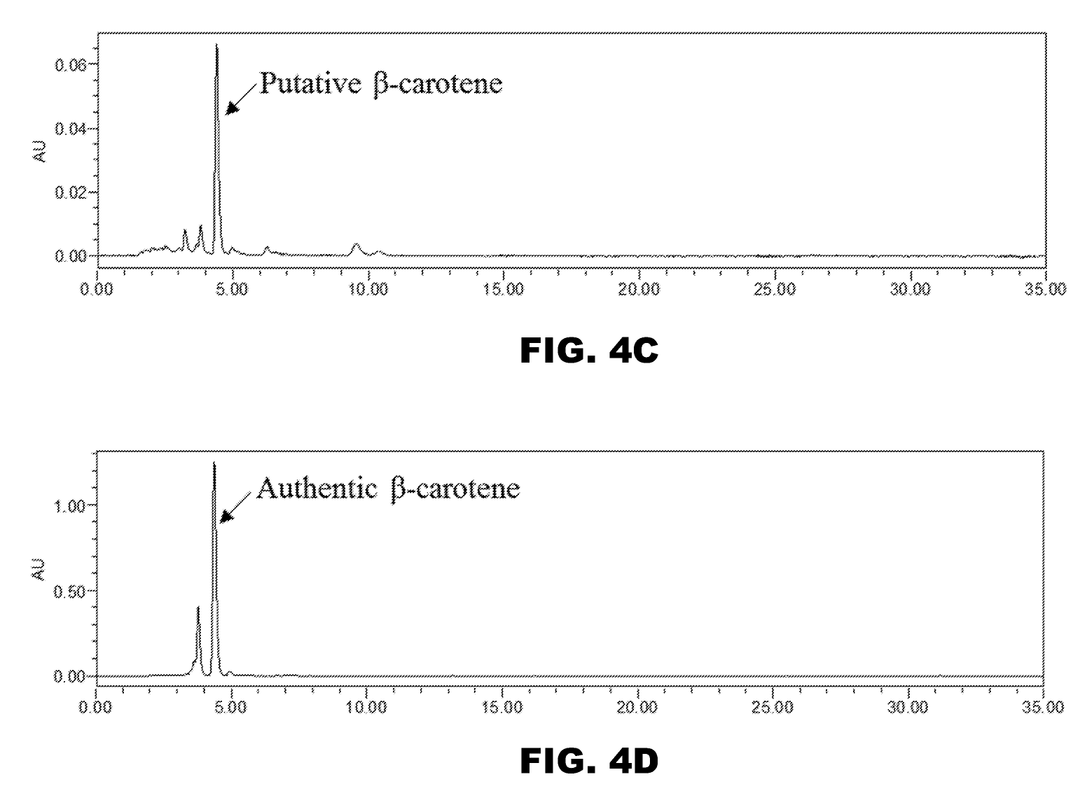

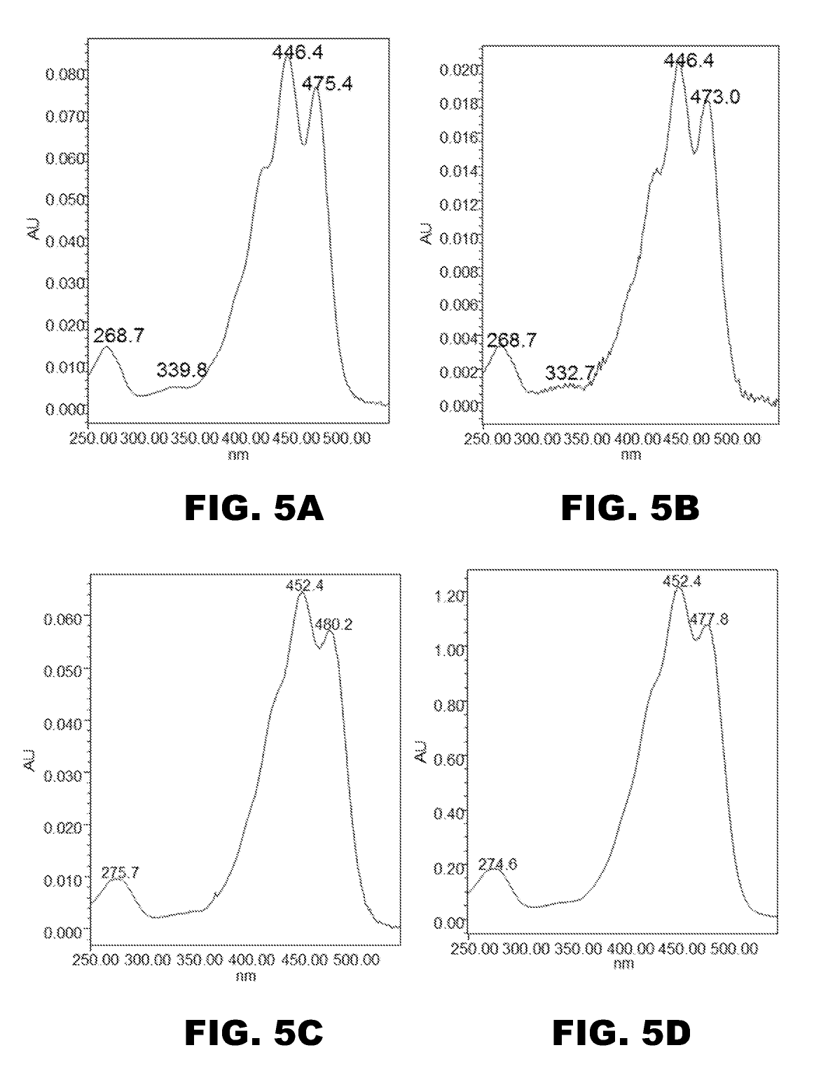

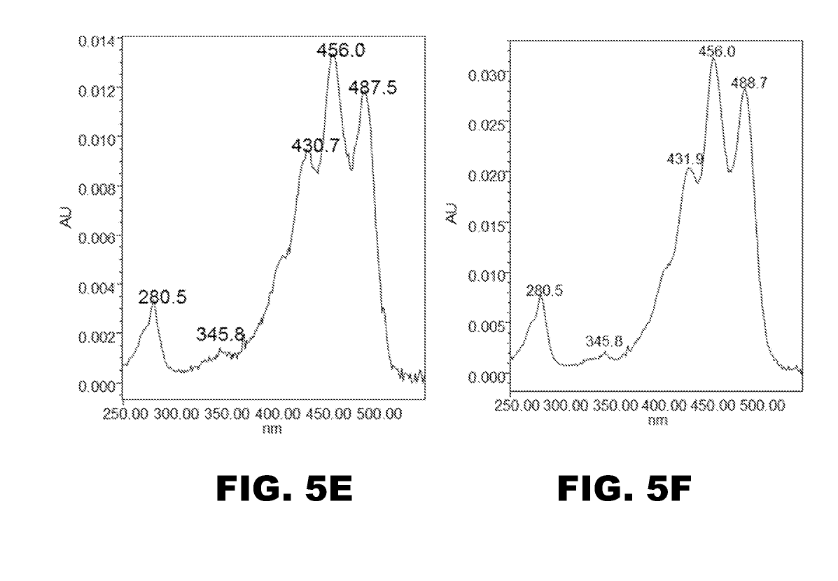

Y. lipolytica strain containing the α-carotene biosynthesis pathway was named as AC-1 and grown in YPD medium. The 200 μl of cell culture was harvested by centrifugation and cell pellet was suspended in 100 μl DMSO for 30 min at 50° C., then 200 μl extraction solvent (Dichloromethane: Methanol (1:3)). The process was repeated three times and the supernatants were pooled for HPLC analysis. The HPLC analysis of α-carotene was performing the same as described for lycopene analysis. When both MpLCYe and CrLCYb or CzLCYb were simultaneously introduced in the lycopene-accumulating Y. lipolytica (LY-2 strain), α-carotene was predominantly produced (52%) (FIGS. 4A, 4B, 4C, 4D). The other three major peaks were tentatively identified as β-carotene (32%), β-carotene (12%) and γ-carotene (4%) by comparison of UV spectrum of authentic β-carotene and the data in the literatures (FIGS. 5A, 5B, 5C, 5D, 5E, 5F). The result indicated that MpLCYe and CrLCYb or CzLCYb activity generates β- and ε-rings from the Ψ end of lycopene, α-carotene, β-carotene, γ-carotene and δ-carotene. But the combination of MpLCYe and MpLCYb can't produce α-carotene.

### Example 7

### Construction of Lutein Biosynthetic Pathway in Yarrowia lipolytica

The conversion of α-carotene to lutein involves two enzymes, β-carotene hydroxylase (CYP97A or BHY) and P450 carotene ε-ring hydroxylase (CYP97C) for β-ring 3-hydroxylation and ε-ring 3′-hydroxylation, respectively (FIG. 1). It has been reported that the carotenoid hydroxylase genes of liverwort Marchantia polymorpha L (SEQ ID NO: 43) and are encoded β-ring hydroxylase and ε-ring 3′-hydroxylation (SEQ ID NO: 46) of α-carotene. The N-terminus amino acids were predicted to be a transit peptide to chloroplast and were removed in yeast expression system. The truncated coding regions of the liverwort tMpBHY (SEQ ID NO: 44) and tMpCYP97C (SEQ ID NO: 47) were synthesized based on Y. lipolytica preferred-codon usage and amplified by tMpBHY-BamHIF (SEQ ID NO: 20) and tMpBHY-AvrIIR (SEQ ID NO: 21), and tMpCYP97C-BamHIF (SEQ ID NO: 22) and tMpCYP97C-AvrIIR (SEQ ID NO: 23), and cloned into the BamHIF/AvrII sites of YAL-zeta-URA3-TEF-XPR2. The two plasmids were named as YAL-zeta-URA3-TEF-MpBHY and YAL-zeta-URA3-TEF-MpCYP97C. Then the TEF-MpBHY-XPR2 cassettes were obtained by PCR amplification and cloned into the SalI/AscI restriction sites of the YAL-zeta-URA3-TEF-MpCYP97C vector to generate the YAL-zeta-URA3-TEF-MpCYP97C-TEF-MpBHY plasmid.

### Example 8

### Construction of Yarrowia lipolytica Strains Producing Lutein

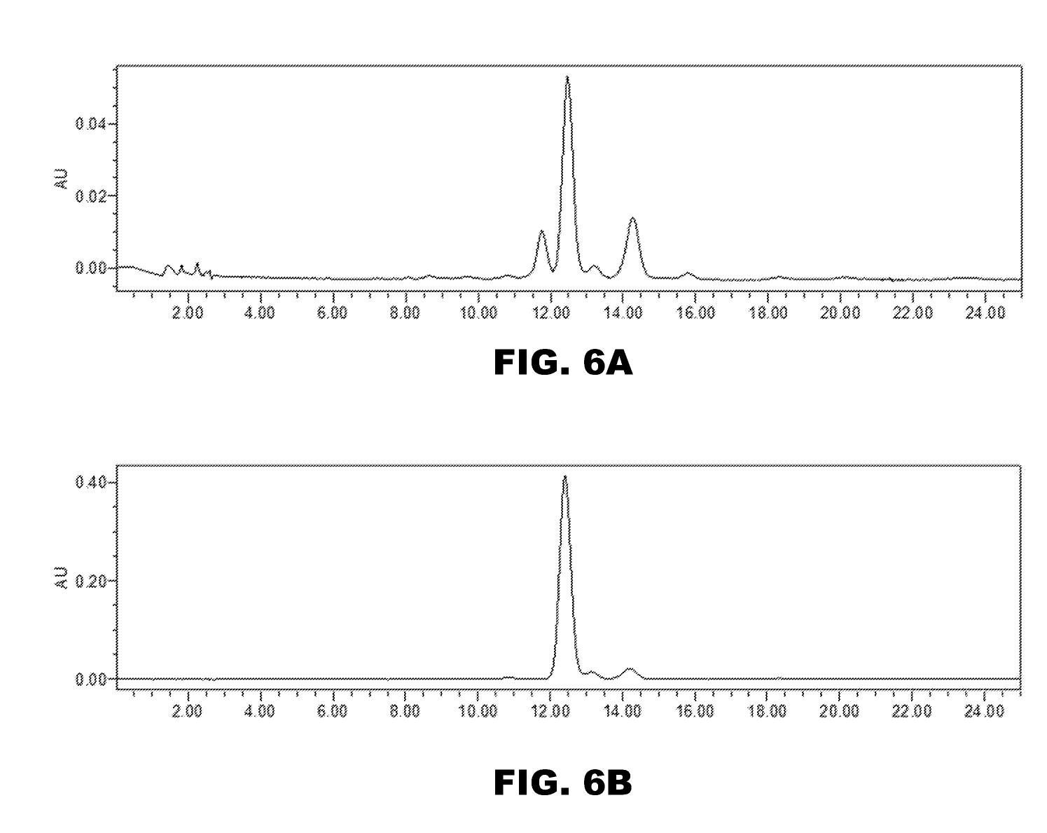

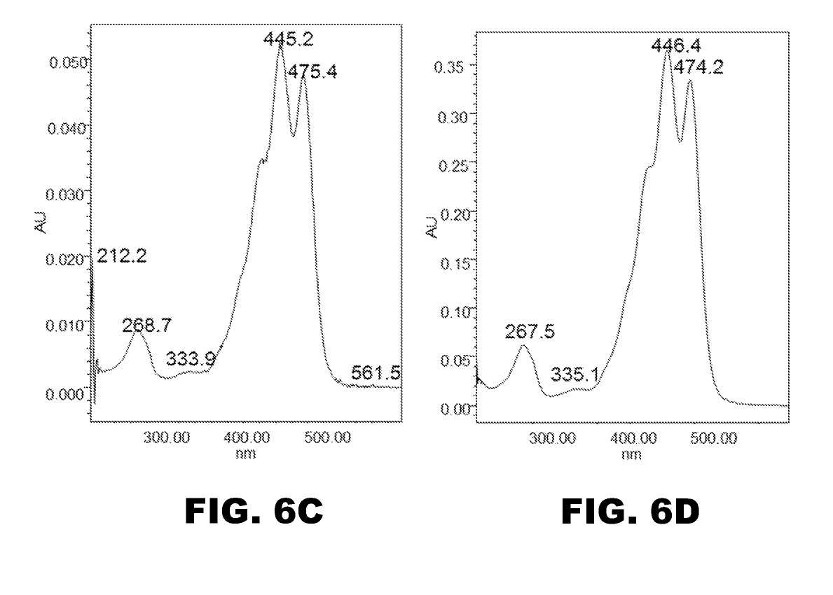

The α-carotene-producing strain AC-1 was transformed plasmid YAL-LEU2-Cre to excise URA3 selection marker and the resulting α-carotene-producing strain without URA3 marker gene was designated AC-2. Plasmid YAL-zeta-URA3-TEF-MpCYP97C-TEF-MpBHY was cut by NotI to extract large fragment containing MpCYP97C-MpBHY cassette. The cassette was introduced into AC-2 strain host and plated on minimal media plate without uracil supplementation. Those red-orange colonies was inoculated into 5 ml YPD medium and extracted for HPLC analysis. Extraction of lutein was same as described above for α-carotene extraction. The extract was collected after centrifugation, and the extraction procedure was repeated three times. The HPLC analysis of lutein was performing the same as described for α-carotene analysis. Another HPLC method was developed for the better separation of lutein. Lutein samples were separated by reverse-phase chromatography on a Develosil RP-Aqueous C30 carotenoid column (particle size 5 μm; 250×4.6 mm) isocratically using a mobile phase of Methanol: Acetonitrile (50:50, v/v) at a flow rate of 1.2 ml/min. Peaks were measured at a wavelength from 250-600 nm to facilitate the detection of lutein. As shown in FIGS. 6A, 6B, 6C, 6D, the engineered strain indeed produced lutein compared with authentic lutein standard by comparing their retention time and UV spectrum.

### Example 9

### Construction of Expression Vectors for β-Cryptoxanthin Production in Yarrowia Lipolytica

For β-carotene biosynthesis, the three-gene expression cassette vector, YAL-zeta-URA3-TEF-OptcarB-TEF-OptcarRP-TEF-GGPPS::FPPS, was generated using the same strategy used for generating the YAL-zeta-URA3-TEF-OptcarB-TEF-OptcarRP*-TEF-GGPPS::FPPS vector described above with the exception that the OptcarRP* gene was replaced with the OptcarRP gene.

The conversion of β-carotene to β-cryptoxanthin needs β-carotene hydroxylase (BCH). This conversion typically leads to the synthesis of zeaxanthin. So, it's necessary to identify enzymes which can convert β-carotene to β-cryptoxanthin efficiently without zeaxanthin accumulation. The different BCH genes from bacteria and plants were screened in Yarrowia lipolytica expression system. The coding sequences of BCH gene from Glycine max showed maximal accumulation of β-cryptoxanthin without zeaxanthin. The β-carotene hydroxylase (GmBCH) (SEQ ID NO: 49) from Glycine max codon-optimized for expression in Yarrowia lipolytica (SEQ ID NO: 50) was synthesized and amplified using primers GmBCH-BamHIF (SEQ ID NO: 24) and GmBCH-AvrIIR (SEQ ID NO: 25), and cloned into BamHI and AvrII restriction sites of YAL-zeta-URA3-TEF-XPR2 vector, to generate YAL-zeta-URA3-TEF-GmBCH.

### Example 10

### Construction of Yarrowia Lipolytica Strains for the Production of β-Cryptoxanthin

Plasmid YAL-zeta-URA3-TEF-OptcarB-TEF-OptcarRP-TEF-FPPS::GGPPS was digested with NotI and the 10 kb fragment was gel purified. The fragment was used to transform Yarrowia lipolytica CLIB138 host and select on minimal media plate without uracil. The yellow colonies were grown in 5 ml YPD medium for 4 days at 30° C. and extracted for further HPLC analysis. The HPLC analysis of β-cryptoxanthin was performing the same as described for lycopene analysis, except a flow rate of 0.5 ml/min was used. The highest colony with β-carotene named as BC-1 and was chosen for further analysis. As shown in FIGS. 4A, 4B, 4C, 4D and FIGS. 5A, 5B, 5C, 5D, 5E, 5F, the Y. lipolytica carrying carRP, carB and FPPS:: GGPPS genes accumulates β-carotene by comparing the retention time (FIGS. 4C and 4D) and UV spectrum (FIGS. 5C and 5D) with authentic β-carotene standard. The highest colony with β-carotene named as BC-1 and was chosen for further analysis.

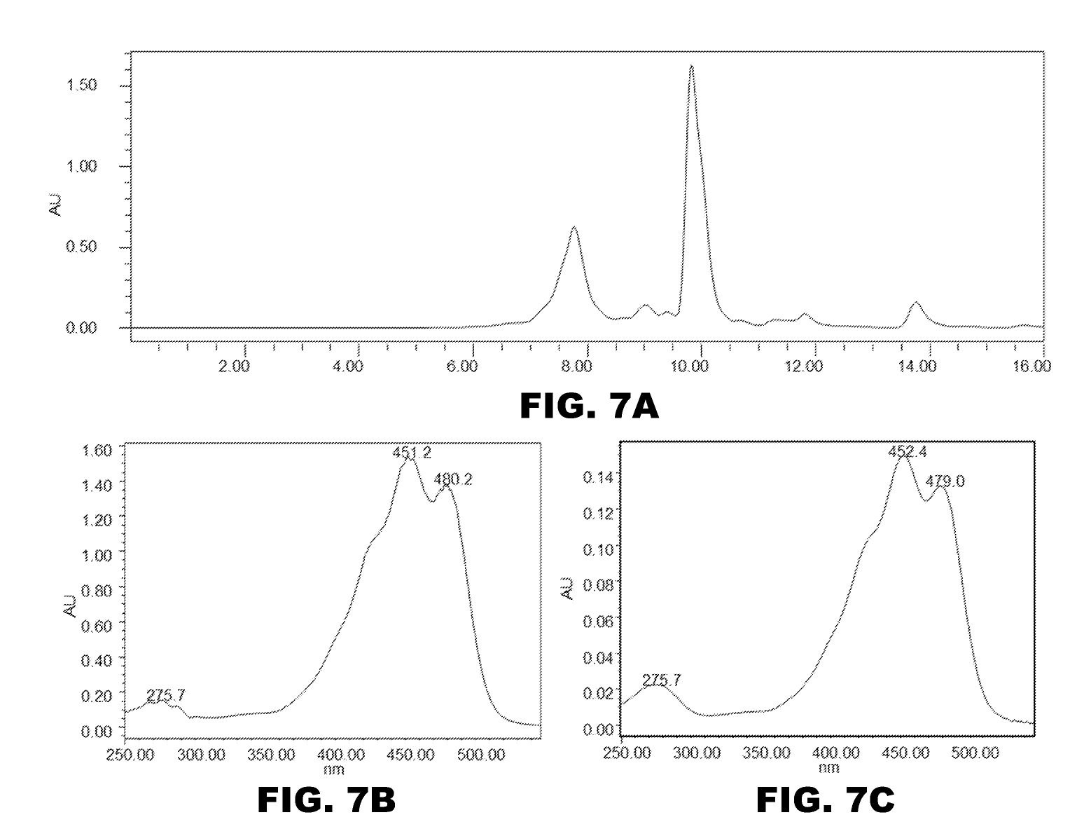

β-carotene-producing strain BC-1 was transformed plasmid YAL-LEU2-Cre to excise URA3 selection marker. The resulting β-carotene-producing strain without URA3 marker gene was designated BC-2. Plasmid YAL-zeta-URA3-TEF-GmBCH was cut by NotI to extract the TEF-GmBCH cassette. The cassette was introduced into BC-2 strain host and plated on minimal media plate without uracil supplementation. Those yellow-orange colonies was inoculated into 5 ml YPD medium and extracted for HPLC analysis. As shown in FIGS. 7A, 7B, 7C, the new peak at 9.83 min is identified as putative β-cryptoxanthin by comparing published data and the peak at 13.75 min is identified as β-carotene by comparing authentic β-carotene.

### Example 11

### Analysis of Putative α-Carotene, Lutein and β-Cryptoxanthin by MaXis Quadrupole Time-Of-Flight (Q-TOF) Mass Spectrometer

The yeast extract samples were analyzed by HPLC to identify the putative carotenoids. The putative purified peaks of α-carotene, lutein and β-cryptoxanthin were analyzed by MaXis quadrupole time-of-flight (Bruker, Bremen, Germany) mass spectrometer to confirm mass (Washington University Biomedical Mass Spectrometry Resource). MS was carried out in the positive ion atmospheric pressure chemical ionization (APCI) ionization mode. The settings were as follows: capillary voltage, 3.5 kV; nebulizer gas, 2 bar; 6 L/min drying gas flow rate and 300° C. dry temperature, and, respectively. Full scan spectra were obtained by scanning masses between m/z 100 and 1000.

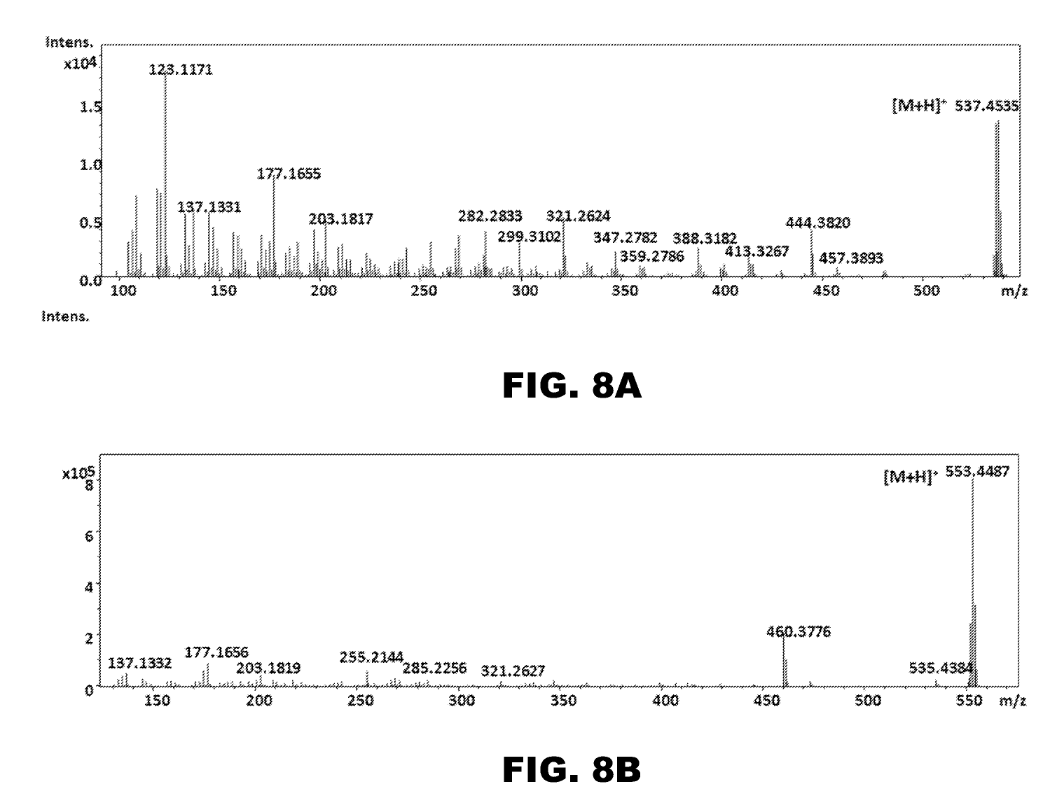

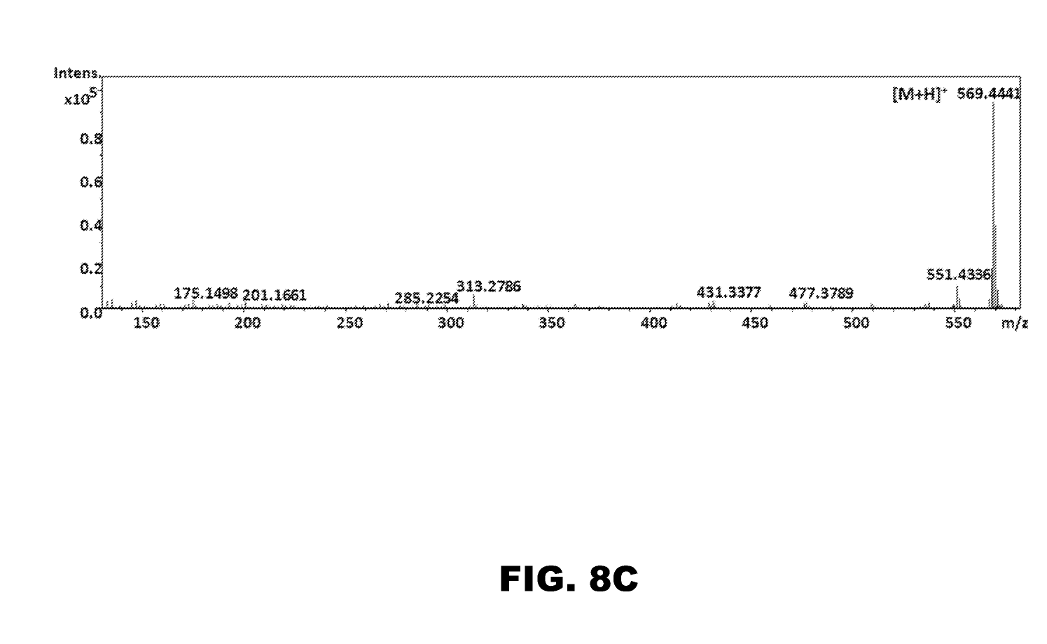

As shown in FIGS. 8A, 8B, 8C MS results, all of three carotenoids ionized by APCI showed the protonated molecular ion [M+H]⁺: 537.4 α-carotene, 569.4 for lutein and 553.4 for β-cryptoxanthin (FIGS. 8A, 8B, 8C). Most of the fragment ion observed in the positive ion APCI product ion tandem mass spectrum of α-carotene (FIG. 8A)(e.g., m/z 457, m/z 444, m/z 413, m/z 137, m/z 123, m/z 177). Lutein is structurally similar to α-carotene except that the rings are hydroxylated. FIG. 8B shows that in the MS spectrum of lutein, the fragments [M+H−18]⁺ at m/z 551 and [M+H−92]⁺ at m/z 477 are abundant ions. β-cryptoxanthin is similar in structure to β-carotene except for the presence of a hydroxyl group on one of the two rings. Elimination of water from the protonated molecule, which is characteristic of hydroxylated xanthophylls, was observed at m/z 535.

### Materials and Methods for Examples 1-11

The Yarrowia lipolytica strain, CLIB138 (MatB, leu2-35, lys5-12, ura3-18, xpr2LYS5), was purchased from CIRM-Levures (Thiverval-grignon, France) and used as host cells in the following exemplifications. All DNA manipulations were performed according to standard procedures. Restriction enzymes and T4 DNA Ligase were purchased from New England Biolabs (Ipswich, Mass.). All PCR amplification and cloning reactions were performed using Phusion® High-Fidelity DNA Polymerase from New England Biolabs (Ipswich, Mass.).

What is claimed:

1. A recombinant microorganism comprising at least one artificial nucleic acid construct comprising:
(a) a nucleic acid comprising a sequence encoding a lycopene ε-cyclase enzyme from Marchantia polymorpha; and
(b) a nucleic acid comprising a sequence encoding a lycopene β-cyclase enzyme selected from a lycopene β-cyclase enzyme from Chlamydomonas reinhardtii, a lycopene β-cyclase enzyme from Chromochloris zofingiensis, and a combination thereof;
wherein the nucleic acid sequences are operably linked to one or more expression control sequences.

2. The recombinant microorganism of claim 1, wherein the microorganism further comprises:
a) a nucleic acid comprising a sequence encoding a β-carotene hydroxylase; and
b) a nucleic acid comprising a sequence encoding a P450 carotene ε-ring hydroxylase;
wherein the nucleic acid sequences are operably linked to one or more expression control sequences.

3. The recombinant microorganism of claim 2, wherein the β-carotene hydroxylase enzyme comprises the β-carotene hydroxylase enzyme from Marchantia polymorpha and the P450 carotene ε-ring hydroxylase enzyme comprises the P450 carotene ε-ring hydroxylase enzyme from Marchantia polymorpha.

4. The recombinant microorganism of claim 3, wherein the microorganism further comprises:
a) a nucleic acid comprising a sequence encoding a phytoene synthase enzyme; and
b) a nucleic acid comprising a sequence encoding a phytoene dehydrogenase enzyme;
wherein the nucleic acid sequences are operably linked to one or more expression control sequences.

5. The recombinant microorganism of claim 4, wherein the phytoene synthase enzyme comprises a lycopene cyclase/phytoene synthase enzyme modified to decrease lycopene cyclase activity, wherein the enzyme is selected the group consisting Mucor circinelloides, Phycomyces blakesleeanus, and Xanthophyllomyces dendrorhous, and wherein the phytoene dehydrogenase enzyme is selected from a phytoene dehydrogenase from Mucor circinelloides, from Xanthophyllomyces dendrorhous, and from Phycomyces blakesleeanus.

6. The recombinant microorganism of claim 1, wherein the microorganism is selected from Yarrowia lipolytica, and Saccharomyces cerevisiae.

7. The recombinant microorganism of claim 1, wherein the microorganism further comprises nucleic acid sequences for producing geranyl geranyl diphosphate.

8. The recombinant microorganism of claim 1, comprising α-carotene.

9. The recombinant microorganism of claim 2, comprising lutein.

10. The recombinant microorganism of claim 1, wherein the nucleic acid expression construct comprising a nucleic acid sequence encoding lycopene ε-cyclase enzyme comprises an amino acid sequence with at least 80% identity to an amino acid sequence of SEQ ID NO: 35.

11. The recombinant microorganism of claim 1, wherein the nucleic acid expression construct comprising a nucleic acid sequence encoding lycopene β-cyclase enzyme comprising an amino acid sequence with at least 80% identity to an amino acid sequence selected from SEQ ID NO: 37 and SEQ ID NO: 41.

12. The recombinant microorganism of claim 1, wherein the nucleic acid expression construct comprising a nucleic acid sequence encoding β-carotene hydroxylase enzyme with at least 80% identity to an amino acid sequence of SEQ ID NO: 44.

13. The recombinant microorganism of claim 1, wherein the nucleic acid expression construct comprising a nucleic acid sequence encoding P450 carotene ε-ring hydroxylase enzyme with at least 80% identity to an amino acid sequence SEQ ID NO: 47.

14. A recombinant microorganism comprising at least one artificial nucleic acid construct comprising:
a) a nucleic acid having a sequence encoding a lycopene cyclase enzyme; and
b) a nucleic acid having a sequence encoding a β-carotene hydroxylase enzyme from Glycine max;
wherein the nucleic acid sequences are operably linked to one or more expression control sequences.

15. The recombinant microorganism of claim 14, wherein the microorganism further comprises:
a) a nucleic acid having a sequence encoding a phytoene synthase enzyme; and
b) a nucleic acid having a sequence encoding a phytoene dehydrogenase activity;
wherein the nucleic acid sequences are operably linked to one or more expression control sequences.

16. The recombinant microorganism of claim 15, wherein the lycopene cyclase enzyme and the phytoene synthase enzyme comprises phytoene synthase and lycopene cyclase of a lycopene cyclase/phytoene synthase from Mucor circinelloides, and the phytoene dehydrogenase activity comprises phytoene dehydrogenase from Mucor circinelloides.

17. The recombinant microorganism of claim 14, wherein the microorganism is selected from Yarrowia lipolytica, and Saccharomyces cerevisiae.

18. The recombinant microorganism of claim 14, wherein the microorganism further comprises nucleic acid sequences for producing geranyl geranyl diphosphate.

19. The recombinant microorganism of claim 14, comprising β-cryptoxanthin.

20. The recombinant microorganism of claim 14, wherein the nucleic acid expression construct comprising a nucleic acid sequence encoding β-carotene hydroxylase enzyme comprising an amino acid sequence with at least 80% identity to an amino acid sequence SEQ ID NO: 50.

21. An artificial nucleic acid expression construct for use in production of a xanthophyll, the nucleic acid encoding a polypeptide comprising an amino acid sequence with at least 80% identity to an amino acid sequence selected from SEQ ID NO: 35, SEQ ID NO: 37 and SEQ ID NO: 41, SEQ ID NO: 44, SEQ ID NO: 47, and SEQ ID NO: 50.

22. A method of producing lutein, the method comprising:
a) providing a recombinant microorganism of claim 2;
b) cultivating the recombinant microorganism under conditions sufficient for the production of lutein; and
c) isolating lutein from the recombinant microorganism.

23. A method of producing β-cryptoxanthin, the method comprising:
a) providing a recombinant microorganism of claim 16;
b) cultivating the recombinant microorganism under conditions sufficient for the production of β-cryptoxanthin; and
c) isolating β-cryptoxanthin from the recombinant microorganism.
